(19) United States
(12) Patent Application Publication
(10) Pub. No.: US 2020/0082908 A1
(43) Pub. Date: Mar. 12, 2020

(54) Methods for Optimizing Direct Targeted Sequencing

(71) Applicant: MYRIAD WOMEN'S HEALTH, INC., South San Francisco, CA (US)

(72) Inventors: Henry Lai, South San Francisco, CA (US); Clement S. Chu, South San Francisco, CA (US)

(21) Appl. No.: 16/558,009

(22) Filed: Aug. 30, 2019

Related U.S. Application Data
(63) Continuation of application No. PCT/US2018/020744, filed on Mar. 2, 2018.
(60) Provisional application No. 62/466,593, filed on Mar. 3, 2017.

(51) Int. Cl. G16B 30/00; C12Q 1/6874; C12N 15/10; G16B 25/00; G16B 40/00

(52) U.S. Cl. CPC G16B 30/00; C12Q 1/6874; C12N 15/1068; G16B 25/00; G16B 40/00; C12N 15/1089

(57) ABSTRACT

Described are methods for selecting an amount of a critical parameter (such as an amount of a sequencing library, amount of a capture probe library, or a number of amplification cycles) for direct targeted sequencing. The methods include hybridizing capture probes in a capture probe library to surface-bound oligonucleotides; extending the surface-bound oligonucleotides using the hybridized capture probes as a template; hybridizing nucleic acid molecules from a sequencing library to the surface-bound capture probes; extending the surface-bound capture probes using the hybridized nucleic acid molecules as a template; amplifying the surface-bound complements of the nucleic acid molecules by bridge amplification for a number of amplification cycles; sequencing the amplified surface-bound complements of the nucleic acid molecules to determine an average cluster density after a predetermined number of sequencing cycles; repeating these steps at a plurality of different amounts of the critical parameter; and selecting an amount of the critical parameter.

### CROSS-REFERENCE TO RELATED APPLICATION

This application claims priority from U.S. Provisional Application Ser. No. 62/466,593, filed on Mar. 3, 2017, the entire contents of which are incorporated herein by reference.

### TECHNICAL FIELD

The present invention relates to methods for the selection an amount of one or more critical parameters by screening for cluster density, cluster intensity, and/or a sequencing quality metric, which allows for the optimization of direct targeted sequencing.

### BACKGROUND

Direct targeted sequencing (DTS) is a method of integrated target capture and sequencing on a single surface, such as a sequencing flow cell. In target capture, capture probes are oligonucleotides that can hybridize to specific target regions of nucleic acid molecules from within a sequencing library. This method enables enrichment of target regions and allows subsequent sequencing efforts to focus on relevant genomic regions or transcripts of interest, for example in deep resequencing to detect rare mutations. By immobilizing capture probes directly on the sequencing surface, direct targeted sequencing enables more efficient high-throughput sequencing of regions of interest. Exemplary methods of direct targeted sequencing are described in U.S. Pat. No. 9,309,556, entitled “Direct Capture, Amplification and Sequencing of Target DNA using Immobilized Primers”, which is hereby incorporated by reference in its entirety. Additional exemplary methods of direct targeted sequencing are described in U.S. Pat. No. 9,092,401, entitled “System and Method for Detecting Genetic Variation”; Myllykangas et al. “Efficient targeted resequencing of human germline and cancer genomes by oligonucleotide-selective sequencing.” Nat Biotechnol. 29(11):1024-7 (2011); and Hopmans et al., “A programmable method for massively parallel targeted sequencing.” Nucleic Acids Res. 42(10):e88 (2014).

Direct targeted sequencing entails first generating surface-bound capture probes. Capture probes from a capture probe library comprise a region that hybridizes onto one population of surface-bound oligonucleotides and another region that comprises the sequence of the target region. Using the hybridized capture probe as a template, surface-bound oligonucleotides are extended to produce surface-bound capture probes that comprise a sequence complementary to a portion of a region of interest. Nucleic acid molecules from a sequencing library are then introduced, and molecules containing the region of interest are hybridized onto the surface-bound capture probes. Using the sequencing library molecules as a template, surface-bound capture probes are extended to produce surface-bound complements of the captured nucleic acid molecules. These surface-bound complements of target nucleic acid molecules are then directly amplified by bridge amplification and sequenced. These methods can be applied to the surface of a sequencing flow cell to capture specific genomic regions of interest from a sample, which is then amplified and directly sequences on the flow cell (Hopmans et al., A programmable method for massively parallel targeted sequencing. Nucleic Acids Res. 42(10):e88 (2014)).

The disclosures of all publications referred to herein are each hereby incorporated herein by reference in their entireties. To the extent that any reference incorporated by references conflicts with the instant disclosure, the instant disclosure shall control.

### SUMMARY

The present invention relates to methods for the selection of an amount of one or more critical parameters (such as an amount of a sequencing library, an amount of a capture probe library, or a number of amplification cycles) by screening for cluster density, cluster intensity, and/or a sequencing quality metric, which allows for the optimization of direct targeted sequencing. The selected amount of the critical parameter can be used to enrich a test sequencing library by direct targeted sequencing, and the enriched sequencing library can be sequenced.

In some embodiments, there is provided a method for selecting an amount of a sequencing library for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different amounts of the sequencing library; and (i) selecting an amount of the sequencing library that provides: (1) the highest average cluster density, wherein the highest average cluster density is within a predetermined cluster density range; (2) an average cluster density that overlaps with a variance of the highest average cluster density, wherein the highest average cluster density and the average cluster density provided by the selected amount of the sequencing library are within a predetermined cluster density range; or (3) a cluster density variance that overlaps with the variance of the highest average cluster density, wherein the highest average cluster density and the average cluster density provided by the selected amount of the sequencing library are within a predetermined cluster density range.

In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the sequencing library is a predetermined percentage of the average cluster density provided by the selected amount of the sequencing library. In some embodiments, the cluster density variance provided by the selected amount of the sequencing library is a predetermined statistical variance of the cluster density provided by the selected amount of the sequencing library.

In some embodiments, the method comprises: determining an average sequencing quality metric after the predetermined number of sequencing cycles; selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within the predetermined cluster density range; and selecting the amount of the sequencing library that provides the highest average sequencing quality metric from the plurality of selected amounts of the sequencing library that provide an average cluster density that overlaps with a variance of the highest average cluster density or a cluster density variance that overlaps with the variance of the highest average cluster density.

In some embodiments, the method comprises: determining an average cluster intensity and an average sequencing quality metric after the predetermined number of sequencing cycles; selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range; selecting a plurality of amounts of the sequencing library that provide an average sequencing quality metric that overlaps with a variance of the highest average sequencing quality metric, or a sequencing quality metric variance that overlaps with the variance of the highest average sequencing quality metric, from the plurality of selected amounts of the sequencing library that provide an average cluster density that overlaps with a variance of the highest average cluster density or a cluster density variance that overlaps with the variance of the highest average cluster density; and selecting the amount of the sequencing library that provides the highest average cluster intensity from the plurality of selected amounts of the sequencing library that provide an average sequencing quality metric that overlaps with a variance of the highest average sequencing quality metric, or a sequencing quality metric variance that overlaps with the variance of the highest average sequencing quality metric.

In some embodiments, the variance of the highest average sequencing quality metric is a predetermined percentage of the highest average sequencing quality metric. In some embodiments, the variance of the highest average sequencing quality metric is a predetermined statistical variance associated with the highest average sequencing quality metric. In some embodiments, the sequencing quality metric variance provided by the selected amount of the sequencing library is a predetermined percentage of the average sequencing quality metric provided by the selected amount of the sequencing library. In some embodiments, the sequencing quality metric variance provided by the selected amount of the sequencing library is a predetermined statistical variance of the sequencing quality metric provided by the selected amount of the sequencing library.

In some embodiments, the sequencing quality metric is a percentage Q30 quality score or a percentage of clusters passing filter.

In some embodiments, the method comprises determining an average cluster intensity after the predetermined number of sequencing cycles; selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range; and selecting an the amount of the sequencing library that provides the highest average cluster intensity from plurality of selected amounts of the sequencing library that provide an average cluster density that overlaps with a variance of the highest average cluster density or a cluster density variance that overlaps with the variance of the highest average cluster density.

In some embodiments of the methods described above, the method further comprises repeating steps (a)-(g) at a plurality of amounts of the capture probe library; and selecting an amount of the capture probe library that provides: (1) the highest average cluster density, wherein the highest average cluster density is within a predetermined cluster density range; (2) an average cluster density that overlaps with a variance of the highest average cluster density, wherein the highest average cluster density and the average cluster density provided by the selected amount of the capture probe library are within a predetermined cluster density range; or (3) a cluster density variance that overlaps with the variance of the highest average cluster density, wherein the highest average cluster density and the average cluster density provided by the selected amount of the capture probe library are within a predetermined cluster density range.

In some embodiments, the amount of the sequencing library and the amount of the capture probe library are selected simultaneously. In some embodiments, the amount of the sequencing library and the amount of the capture probe library are selected sequentially.

In some embodiments, the method comprises determining an average sequencing quality metric after the predetermined number of sequencing cycles; selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and selecting the amount of the capture probe library that provides the highest average sequencing quality metric from the plurality of selected amounts of the capture library that provide an average cluster density that overlaps with the variance of the highest average cluster density or a cluster density variance that overlaps with the variance of the highest average cluster density.

In some embodiments, the method comprises determining an average sequencing quality metric and an average cluster intensity after the predetermined number of sequencing cycles; selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture library that provide an average cluster density that overlaps with the variance of the highest average cluster density or a cluster density variance that overlaps with the variance of the highest average cluster density; and selecting the amount of the capture probe library that provides the highest average cluster intensity from the plurality of amounts of the capture probe library that provide an average sequencing quality metric that overlaps with a variance of the highest average sequencing quality metric, or a sequencing quality metric variance that overlaps with the variance of the highest average sequencing quality metric.

In some embodiments, the method comprises determining an average cluster intensity after the predetermined number of sequencing cycles; selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and selecting the amount of the capture probe library that provides the highest average cluster intensity from the plurality of selected amounts of the capture library that provide an average cluster density that overlaps with the variance of the highest average cluster density or a cluster density variance that overlaps with the variance of the highest average cluster density.

In some embodiments of the methods described above, the method comprises repeating steps (a)-(g) at a plurality different numbers of amplification cycles; and selecting the number of amplification cycles that provides: (1) the highest average cluster density, wherein the highest average cluster density is within a predetermined cluster density range; (2) an average cluster density that overlaps with a variance of the highest average cluster density, wherein the highest average cluster density and the average cluster density provided by the selected number of amplification cycles are within a predetermined cluster density range; or (3) a cluster density variance that overlaps with the variance of the highest average cluster density, wherein the highest average cluster density and the average cluster density provided by the selected number of amplification cycles are within a predetermined cluster density range.

In some embodiments, the sequencing library and the number of amplification cycles are selected simultaneously. In some embodiments, the amount of the sequencing library and the number of amplification cycles are selected sequentially. In some embodiments, the amount of the sequencing library, amount of the capture probe library, and number of amplification cycles are selected simultaneously. In some embodiments, the amount of the sequencing library, the amount of the capture probe library, and the number of amplification cycles are selected sequentially.

In some embodiments, the method comprises determining an average sequencing quality metric after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and selecting the number of amplification cycles that provides the highest average sequencing quality metric from the plurality of selected amounts of the capture library that provide an average cluster density that overlaps with the variance of the highest average cluster density or a cluster density variance that overlaps with the variance of the highest average cluster density.

In some embodiments, the method comprises determining an average cluster intensity after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

In some embodiments, the method comprises determining an average cluster intensity and an average sequencing quality metric after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

In some embodiments, there is provided a method for selecting an amount of a capture probe library for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine a cluster density after a predetermined number of sequencing cycles; (h) repeating steps (a)-(g) at a plurality of different amounts of the capture probe library; and (i) selecting an amount of the capture probe library that provides: (1) the highest average cluster density, wherein the highest average cluster density is within a predetermined cluster density range; (2) an average cluster density that overlaps with a variance of the highest average cluster density, wherein the highest average cluster density and the average cluster density provided by the selected amount of the capture probe library are within a predetermined cluster density range; or (3) a cluster density variance that overlaps with the variance of the highest average cluster density, wherein the highest average cluster density and the average cluster density provided by the selected amount of the capture probe library are within a predetermined cluster density range.

In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the capture probe library is a predetermined percentage of the average cluster density provided by the selected amount of the capture probe library. In some embodiments, the cluster density variance provided by the selected amount of the capture probe library is a predetermined statistical variance of the cluster density provided by the selected amount of the capture probe library.

In some embodiments, the method comprises determining an average sequencing quality metric after the predetermined number of sequencing cycles; selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and selecting the amount of the capture probe library that provides the highest average sequencing quality metric from the plurality of selected amounts of the capture library that provide an average cluster density that overlaps with the variance of the highest average cluster density or a cluster density variance that overlaps with the variance of the highest average cluster density.

In some embodiments, the method comprises determining an average sequencing quality metric and an average cluster intensity after the predetermined number of sequencing cycles; selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture library that provide an average cluster density that overlaps with the variance of the highest average cluster density or a cluster density variance that overlaps with the variance of the highest average cluster density; and selecting the amount of the capture probe library that provides the highest average cluster intensity from the plurality of amounts of the capture probe library that provide an average sequencing quality metric that overlaps with a variance of the highest average sequencing quality metric, or a sequencing quality metric variance that overlaps with the variance of the highest average sequencing quality metric.

In some embodiments, the method comprises determining an average cluster intensity after the predetermined number of sequencing cycles; electing a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and selecting the amount of the capture probe library that provides the highest average cluster intensity from the plurality of selected amounts of the capture library that provide an average cluster density that overlaps with the variance of the highest average cluster density or a cluster density variance that overlaps with the variance of the highest average cluster density.

In some embodiments, the method further comprises repeating steps (a)-(g) at a plurality different numbers of amplification cycles; and selecting the number of amplification cycles that provides: (1) the highest average cluster density, wherein the highest average cluster density is within a predetermined cluster density range; (2) an average cluster density that overlaps with a variance of the highest average cluster density, wherein the highest average cluster density and the average cluster density provided by the selected number of amplification cycles are within a predetermined cluster density range; or (3) a cluster density variance that overlaps with the variance of the highest average cluster density, wherein the highest average cluster density and the average cluster density provided by the selected number of amplification cycles are within a predetermined cluster density range.

In some embodiments, the amounts of the capture probe library and the number of amplification cycles are selected simultaneously. In some embodiments, the amount of the capture probe library and the number of amplification cycles are selected sequentially.

In some embodiments, the method comprises determining an average sequencing quality metric after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and selecting the number of amplification cycles that provides the highest average sequencing quality metric from the plurality of selected numbers of amplification cycles that provide an average cluster density that overlaps with the variance of the highest average cluster density or a cluster density variance that overlaps with the variance of the highest average cluster density.

In some embodiments, the method comprises determining an average cluster intensity after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

In some embodiments, the method comprises determining an average cluster intensity and an average sequencing quality metric after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

In some embodiments, there is provided a method for selecting a number of amplification cycles for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine a cluster density after a predetermined number of sequencing cycles; (h) repeating steps (a)-(g) at a plurality of different numbers of amplification cycles; and (i) selecting a number of amplification cycles that provides: (1) the highest average cluster density, wherein the highest average cluster density is within a predetermined cluster density range; (2) an average cluster density that overlaps with a variance of the highest average cluster density, wherein the highest average cluster density and the average cluster density provided by the selected number of amplification cycles are within a predetermined cluster density range; or (3) a cluster density variance that overlaps with the variance of the highest average cluster density, wherein the highest average cluster density and the average cluster density provided by the selected number of amplification cycles are within a predetermined cluster density range.

In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected number of sequencing cycles is a predetermined percentage of the average cluster density provided by the selected number of sequencing cycles. In some embodiments, the cluster density variance provided by the selected number of sequencing cycles is a predetermined statistical variance of the cluster density provided by the selected number of sequencing cycles.

In some embodiments, the method comprises determining an average sequencing quality metric after the predetermined number of sequencing cycles; and selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and selecting the number of amplification cycles that provides the highest average sequencing quality metric from the plurality of selected numbers of amplification cycles that provide an average cluster density that overlaps with the variance of the highest average cluster density or a cluster density variance that overlaps with the variance of the highest average cluster density.

In some embodiments, the method comprises determining an average cluster intensity after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

In some embodiments, the method comprises determining an average cluster intensity and an average sequencing quality metric after the predetermined number of sequencing cycles; selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

In some embodiments, the sequencing quality metric is a percentage Q30 quality score or a percentage of clusters passing filter.

In some embodiments of any of the methods described above, the method further comprises sequencing a sequencing library by direct targeted sequencing using the selected amount of the sequencing library, the selected amount of the capture probe library, or the selected number of amplification cycles.

In some embodiments, there is provided a method of sequencing a test sequencing library, comprising: (a) hybridizing capture probes to surface-bound oligonucleotides, the capture probes comprising a first end comprising a sequence that hybridizes to the first population of surface-bound oligonucleotides and a second end comprising a sequence that hybridizes to a portion of a region of interest, wherein the concentration of the capture probes is about 40 to about 70 nM; (b) extending the surface-bound oligonucleotides using the hybridized capture probes as a template to produce surface-bound capture probes; (c) removing the capture probes; (d) hybridizing nucleic acid molecules from about 5 μM to about 10 μM of the test sequencing library comprising the region of interest to the surface-bound capture probes, wherein the concentration of the nucleic acid molecules results in a cluster density of about 600 K/mm² to about 1500 K/mm²; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for at least 30 amplification cycles; (g) sequencing the amplified surface-bound complements of the nucleic acid molecules.

### BRIEF DESCRIPTION OF THE DRAWINGS

FIG. 1A-1D represents exemplary embodiments of methods for selecting an amount of a critical parameter for direct targeted sequencing. The FIG. 1A depicts an exemplary method for selecting an amount of a critical parameter based on a determined average cluster density. FIG. 1B depicts an exemplary method for selecting an amount of a critical parameter based on a determined average cluster density and an average cluster intensity. FIG. 1C illustrates an exemplary method for selecting an amount of a critical parameter based on a determined average cluster density and a determined average sequencing quality metric. FIG. 1D depicts an exemplary method for selecting an amount of a critical parameter based on a determined average cluster density, a determined average sequencing quality metric, and a determined average cluster intensity.

FIG. 2 illustrates the method of sequencing a sequencing library using direct targeted sequencing, which comprises (a) hybridizing capture probes from a capture probe library to surface-bound oligonucleotides; (b) extending surface-bound oligonucleotides to produce surface-bound capture probes; (c) removing capture probes; (d) hybridizing nucleic acids from a sequencing library to surface-bound capture probes; (e) extending surface-bound capture probes to produce surface-bound complements of nucleic acids; (f) bridge amplification for a number of amplification cycles; and (g) sequencing of amplified surface-bound complements of nucleic acids. Methods of direct targeted sequencing are also described in U.S. Pat. No. 9,092,401, entitled “System and Method for Detecting Genetic Variation”; Myllykangas et al. “Efficient targeted resequencing of human germline and cancer genomes by oligonucleotide-selective sequencing.” Nat Biotechnol. 29(11):1024-7 (2011); and Hopmans et al., “A programmable method for massively parallel targeted sequencing.” Nucleic Acids Res. 42(10):e88 (2014).

### DETAILED DESCRIPTION

Selection of the amounts of critical parameters (such as capture probe library amount, sequencing library amount, or the number of amplification cycles) optimizes sequencing of a test sequencing library using direct targeted sequencing. Direct targeted sequencing (DTS), also referred to as oligonucleotide-selective sequencing (Os-Seq), is a method of integrated target capture and high throughput sequencing on a single surface, such as a sequencing flow cell. DTS generally involves hybridizing capture probes (which include a portion of a region of interest) to surface-bound oligonucleotides, extending the surface-bound oligonucleotides using the hybridized capture probes as a template to generate surface-bound capture probes, hybridizing nucleic acids in a sequence library to the surface-bound capture probes, and extending the surface-bound capture probes using the hybridized capture probes as a template to produce surface-bound complements of the of the nucleic acid molecules. The surface-bound complements are then amplified (by bridge amplification) and subjected to sequencing analysis.

The need to simultaneously achieve efficient target capture and cluster generation for sequencing in carrying out DTS presents unique challenges. The pre-amplified surface bound complements can serve as origin molecules for clusters, and the more pre-amplified surface bound complements on the surface results in a higher cluster density. Bridge amplification relies on surface-bound oligonucleotides that did not were not transformed into surface-bound capture probes. Therefore, too high of a cluster density results in poor bridge amplification and clusters that are smaller than desired, which results in poor average cluster intensity. Too low of a cluster density, however, results in an insufficient diversity of sequencing data, limiting thorough sequencing of the test sequencing library. Multiple parameters can influence the quality of the sequencing data generated by sequencing a test sequencing library which has been enriched by direct targeted sequencing. These parameters can include, but are not limited to, the number and arrangement of surface oligonucleotides, capture probe design, capture probe length, capture probe amount, number of capture probes in a library, variability of capture probes in a library, capture probe hybridization conditions, sequencing library hybridization conditions (time, temperature, chemistry, etc.), sequencing library amount, sequencing library diversity (the proportion of each nucleotide in each position on a template library), sequencing library quality (e.g., contaminating spurious library products such as adapter and primer dimer), sequencing library preparation (e.g., end repair, A-tailing, adaptor ligation, etc.), sequencing library size, sequencing library source, region of interest sequence, region of interest GC content, number of bridge amplification cycles, sequencing platform, sequencing mode, and sequencing chemistry.

The present invention is based on the finding that a small set of parameters (hereinafter also referred to collectively as “critical parameters”), namely, the amount of the sequencing library, the amount of capture probe library, and the number of amplification cycles, are critical for efficient DTS methodology. By varying one or a combination of these critical parameters, sometimes for example at amounts that are significantly higher than those typically used in carrying out DTS, one would arrive at a condition that allows for efficient DTS.

Described herein is a method for selecting an amount of a critical parameter (such as an amount of a sequencing library, and amount of a capture probe library, or a number of amplification cycles) for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different amounts of the critical parameter; and (i) selecting an amount of the critical parameter that provides: (1) the highest average cluster density, (2) an average cluster density that overlaps with a variance of the highest average cluster density, or (3) a cluster density variance that overlaps with the variance of the highest average cluster density, wherein the highest average cluster density and the average cluster density provided by the selected amount of the critical parameter are within a predetermined cluster density range. In some embodiments, an amount for two or more (such as three) critical parameters are selected, which may be selected sequentially or in combination.

In some embodiments, the method further comprises determining an average sequencing quality metric after the predetermined number of sequencing cycles; selecting a plurality of amounts of the critical parameter that provide an average cluster density that overlaps with a variance of the highest average cluster density, or a cluster density variance that overlaps with the variance of the highest average cluster density, wherein the highest average cluster density and the average cluster densities provided by the plurality of selected amounts of the critical parameter are within the predetermined cluster density range; and selecting the amount of the critical parameter that provides the highest average sequencing quality metric from the plurality of selected amounts of the critical that provide an average cluster density that overlaps with a variance of the highest average cluster density or a cluster density variance that overlaps with the variance of the highest average cluster density.

In some embodiments, the method further comprises determining an average cluster intensity and an average sequencing quality metric after the predetermined number of sequencing cycles; selecting a plurality of amounts of the critical parameter that provide an average cluster density that overlaps with a variance of the highest average cluster density, or a cluster density variance that overlaps with the variance of the highest average cluster density, wherein the highest average cluster density and the average cluster densities provided by the plurality of selected amounts of the critical parameter are within a predetermined cluster density range; selecting a plurality of amounts of the critical parameter that provide an average sequencing quality metric that overlaps with a variance of the highest average sequencing quality metric, or a sequencing quality metric variance that overlaps with the variance of the highest average sequencing quality metric, from the plurality of selected amounts of the critical parameter that provide an average cluster density that overlaps with a variance of the highest average cluster density or a cluster density variance that overlaps with the variance of the highest average cluster density; and selecting the amount of the critical parameter that provides the highest average cluster intensity from the plurality of selected amounts of the critical parameter that provide an average sequencing quality metric that overlaps with a variance of the highest average sequencing quality metric, or a sequencing quality metric variance that overlaps with the variance of the highest average sequencing quality metric.

In some embodiments, the method further comprises determining an average cluster intensity after the predetermined number of sequencing cycles; selecting a plurality of amounts of the critical parameter that provide an average cluster density that overlaps with a variance of the highest average cluster density, or a cluster density variance that overlaps with the variance of the highest average cluster density, wherein the highest average cluster density and the average cluster densities provided by the plurality of selected amounts of the critical parameter are within a predetermined cluster density range; and selecting an the amount of the critical parameter that provides the highest average cluster intensity from plurality of selected amounts of the critical parameter that provide an average cluster density that overlaps with a variance of the highest average cluster density or a cluster density variance that overlaps with the variance of the highest average cluster density.

### Definitions

As used herein, the singular forms “a,” “an,” and “the” include the plural reference unless the context clearly dictates otherwise.

Reference to “about” or “approximately” a value or parameter herein includes (and describes) variations that are directed to that value or parameter per se. For example, description referring to “about X” includes description of “X.”

The term “average” as used herein refers to either a mean or a median, or any value used to approximate the mean or the median, unless the context clearly indicates otherwise.

It is understood that aspects and variations of the invention described herein include “consisting” and/or “consisting essentially of” aspects and variations.

The term “oligonucleotide” as used herein denotes a single-stranded deoxyribonucleotide or ribonucleotide. For the purposes of the present disclosure, these terms are not to be construed as limiting with respect to the length of a polymer. The terms can encompass known analogues of natural nucleotides, as well as nucleotides that are modified in the base, sugar and/or phosphate moieties. Oligonucleotides may be synthetic or may be made enzymatically.

The term “capture probe” refers to a single stranded nucleic acid comprising a region or regions that are complementary to a target nucleic acid sequence. A “capture probe” can hybridize to a target nucleic acid sequence by the formation of hydrogen bonds between the complementary bases. The capture probe can be DNA, RNA, or a nucleic acid analogue.

“Cluster density” is the number of discrete clonal nucleic acid clusters per unit of area. Cluster density can be measured in thousands of clusters per square millimeter (“K/mm²”) or thousands of clusters per square millimeter per tile.

“Chastity filter” is a quality control measure utilized by Illumina to determine acceptance or rejection of individual clusters. This filter is typically applied after the first 25 sequencing cycles. The highest intensity base incorporated into a cluster is recorded and its intensity is compared to the next highest fluorescent base recorded for the cluster. This information is used to calculate the chastity filter ratio, which is derived by taking the fluorescence of the highest fluorescent intensity base and dividing it by the fluorescence of the same highest fluorescent intensity base plus the fluorescence of the next highest fluorescence intensity base. Generally, a ratio of 0.6 or greater is considered a “passing” ratio. The chastity filter can remove clusters of low uniformity. Chastity=Highest Intensity/(Highest Intensity+Next Highest Intensity) for each cycle

The “quality score,” or “Q score,” is Q=−10 log₁₀(e), where e is the error probability, or the estimated probability of an erroneous base call. The Q score is logarithmically related to error probability (e) and is conceptually analogous to the Phred quality score used in Sanger sequencing.

The “% Q30” is the number of bases with a “Q score” of 30 or higher. In general, a “% Q” followed by a number is the percent of bases with a quality score of that number or higher. For example, bases with Q20 and Q30 scores have a 1:100 and 1:1000 probability of being called incorrectly.

Median Q-Score, which is defined as the median quality score for each tile over all bases for the current sequencing cycle.

“% Intensity” is the corresponding intensity statistic at a predetermined sequencing cycle as a percentage of that value at the first cycle (i.e. 100%×(intensity at cycle 20)/(intensity at cycle 1)).

“Corrected Intensity” is the intensity corrected for cross-talk between the color channels and phasing and prephasing.

“Called Intensity” is defined as the intensity for the called base (the base, or nucleotide, identified from the data generated by the automated sequencing instrument.

The term “tile” refers to a portion of a sequencing flow cell, wherein each tile has a reference location in the flow cell.

A “variance” refers to a range of values of some distance away from a set value, such as an average or a maximum. The term “variance” includes a “statistical variance” or a predetermined percentage (for example, in reference to an average) or a range at or above a percentile (for example, in reference to a maximum or highest value). A “statistical variance” refers to any value that measures the spread of a distribution including, but not limited to, a standard deviation, a dispersion, or an interquartile range.

It is to be understood that one, some or all of the properties of the various embodiments described herein may be combined to form other embodiments of the present invention. The section headings used herein are for organizational purposes only and are not to be construed as limiting the subject matter described.

### Methods of the Present Invention

The critical parameters (e.g., the amount of the sequencing library, the amount of the capture probe library, or the number of amplification cycles) selected for optimized direct targeted sequencing can be selected based on one or more sequencing metrics. For example, the critical parameters can be selected based on an average cluster density; an average cluster density and an average cluster intensity; an average cluster density and an average sequencing quality metric (such as a percentage Q30 quality score or a percentage of clusters passing filter); or an average cluster density, an average sequencing quality metric, and an average cluster intensity. Further, the amounts of one or more, two or more, or three or more critical parameters can be selected using the methods described herein, either sequentially or in combination.

The plurality of amounts of the critical parameter can be 2 or more, 3 or more, 5 or more, 10 or more, 25 or more, or 50 or more different amounts. In some embodiments, the amounts are within a predetermined range (e.g., a range of amounts of the sequencing library, a range of amounts of the capture probe library, or a range of a number of amplification cycles). In some embodiments, the different amounts are evenly spaced or approximately evenly spaced within the range. In some embodiments, the different amounts are unevenly spaced within the range.

Selecting a Critical Parameter Based on an Average Cluster Density

In one aspect, there is provided a method for selecting an amount of a critical parameter (such as an amount of a sequencing library, and amount of a capture probe library, and/or a number of amplification cycles) for direct targeted sequencing, comprising sequencing a sequencing library enriched by direct targeted sequencing at a plurality of different amounts of the critical parameter to determine an average cluster density after a predetermined number of sequencing cycles for each critical parameter amount; and selecting an amount of the critical parameter that provides the highest average cluster density, wherein the highest average cluster density within a predetermined cluster density range.

For each amount of the critical parameter, an average cluster density is determined. The cluster density is determined as an average because the cluster density may not be uniform across the entire surface. In some embodiments, a cluster density distribution is determined, which can include an average cluster density and a statistical variance. The selected amount of the critical parameter need not be (and is often not) the amount of the critical parameter that provides the highest average cluster density. Too high of a cluster density can result in poor average cluster intensity, which degrades the quality of the sequencing data. Instead, a predetermined cluster density range is selected, and the amount of the critical parameter selected is the amount that provides the highest average cluster density within the predetermined cluster density range. The predetermined cluster density range is selected based on the type of sequencer or surface used, and is generally indicated by the manufacturer of the sequencer or surface, or can be determined by a person of skill in the art.

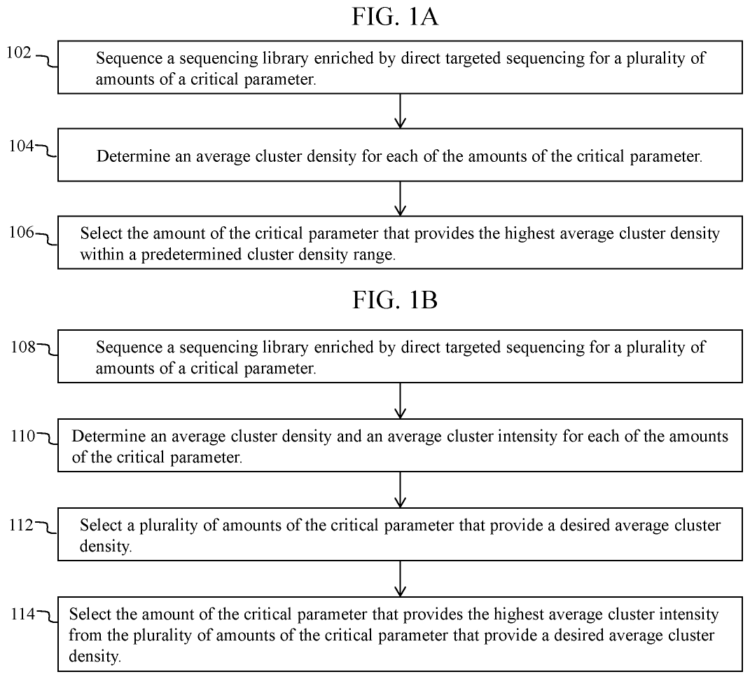

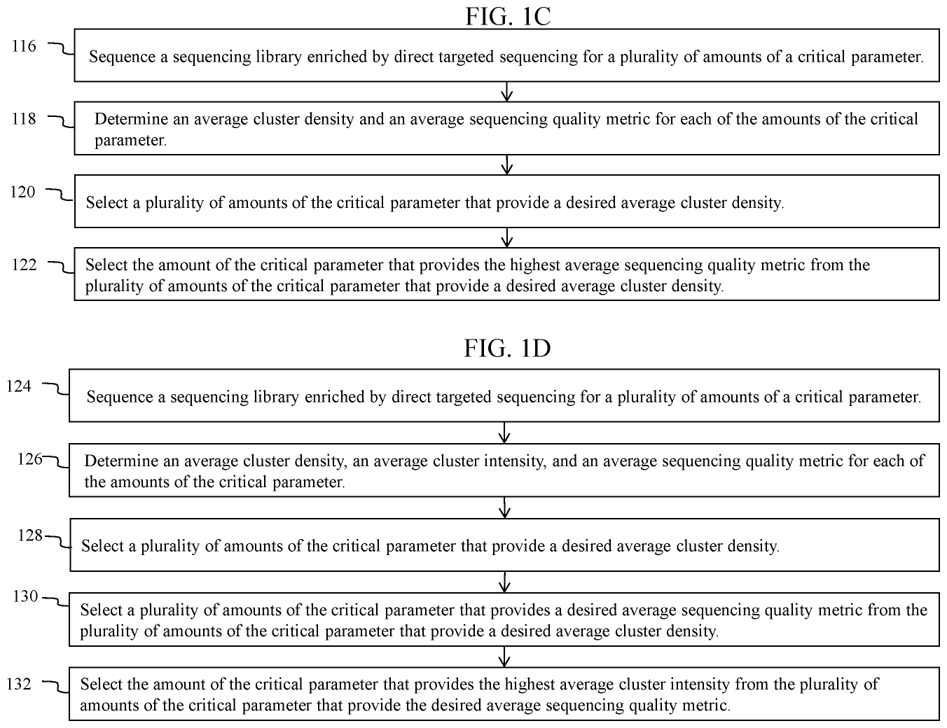

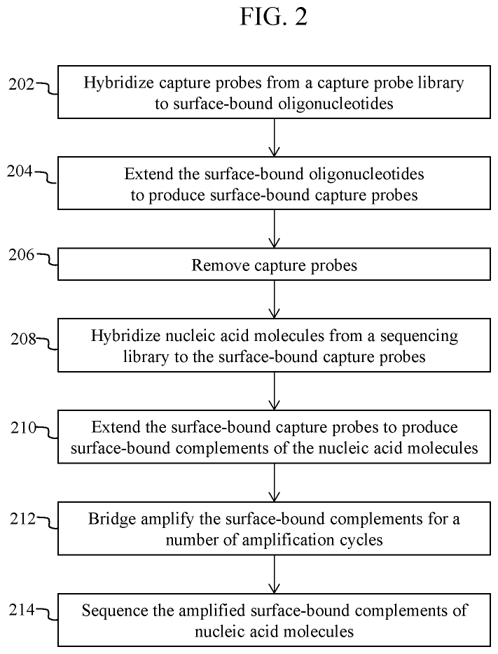

FIG. 1A illustrates a method for selecting an amount of a critical parameter for direct targeted sequencing based on a determined average cluster density after a predetermined number of sequencing cycles. At step 102, a sequencing library enriched by direct targeted sequencing is sequenced for a plurality of amounts of a critical parameter (such as different amounts of a sequencing library, different amounts of a capture probe library, or different numbers of amplification cycles). At step 104, the average cluster density is determined for each of the amounts of the critical parameter. At step 106, the amount of the critical parameter that provides the highest average cluster density within a predetermined cluster density range is selected.

### Selecting a Critical Parameter Based on an Average Cluster Density and an Average Cluster Intensity

In some embodiments, one more critical parameters are selected based on cluster density and an average cluster intensity. A plurality of amounts of the critical parameter are selected based on a desired cluster density; and from the plurality of amounts of the critical parameter selected based on the desired cluster density, an amount of a critical parameter is selected based on an average cluster intensity. For example, in some embodiments, there is a method for selecting an amount of a critical parameter (such as an amount of a sequencing library, and amount of a capture probe library, or a number of amplification cycles) for direct targeted sequencing, comprising sequencing a sequencing library enriched by direct targeted sequencing at a plurality of different amounts of the critical parameter to determine an average cluster density and an average cluster intensity after a predetermined number of sequencing cycles for each critical parameter amount; selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range; and selecting an the amount of the sequencing library that provides the highest average cluster intensity from the plurality of selected amounts of the sequencing library that provide an average cluster density that overlaps with a variance of the highest average cluster density or a cluster density variance that overlaps with the variance of the highest average cluster density.

For each amount of the critical parameter, an average cluster density is determined. The highest average cluster density within the predetermined cluster density range is then determined. The highest average cluster density is associated with a variance. From those amounts of the critical parameter that provide an average cluster density that overlaps with a variance of the highest average cluster density, or provide a cluster density variance that overlaps with the variance of the highest average cluster density, an amount of the critical parameter can be selected that provides the highest average cluster intensity. In some embodiments, the variance is a statistical variance (e.g., a standard deviation, interquartile range, a statistical dispersion, or other statistical variance). The statistical variance can be determined, for example, based on the cluster density variation on the surface for the amount of the critical parameter. For example, some surfaces include a plurality of tiles, and a cluster density is determined for each tile. A statistical variance can be determined for the amount of the critical parameter that provided the highest average cluster density from the cluster density variance of the tiles. In some embodiments, the variance is percentage of (e.g., within 5% or less, within 10% or less, within 15% or less, or within 20% or less) the determined highest average cluster density. In some embodiments, the variance is a percentile (for example, 70th percentile or above, 80th percentile or above, or 90th percentile or above) for the average cluster densities in the pluralities of amounts of the critical parameters. In some embodiments, the selected plurality of amounts of the critical parameter provide an average cluster density that overlaps with the variance of the highest average cluster density (that is, the average cluster density provided by each of the selected amounts of the critical parameter are within the variance (e.g., statistical variance, percentage of, or percentile) of the highest average cluster density). In some embodiments, the selected plurality of amounts of the critical parameter have a variance (e.g., a statistical variance or a percentage of) associated with the determined average cluster density, and that variance overlaps the variance associated with the highest average cluster density. The variances need not fully overlap as long as some portion of the variances overlap. The selected amounts of the critical parameter, each provide an average cluster density (including the highest average cluster density) within the predetermined cluster density range.

From the plurality of amounts of the critical parameter that provide an average cluster density that overlaps with a variance of the highest average cluster density or a cluster density variance that overlaps with the variance of the highest average cluster density (as long as the average cluster density for the plurality of amounts of the critical parameter is within the predetermined cluster density range), an amount of the critical parameter is selected that provides the highest average cluster intensity. The average cluster intensity is determined for at least the amounts of the critical parameter in the plurality of amounts of the critical parameter that provide an average cluster density that overlaps with a variance of the highest average cluster density or a cluster density variance that overlaps with the variance of the highest average cluster density, although in some embodiments the average cluster intensity is determined for each amount of the critical parameter for which an average cluster density was determined.

FIG. 1B illustrates a method for selecting an amount of a critical parameter for direct targeted sequencing based on an average cluster density and an average cluster intensity after a predetermined number of sequencing cycles. At step 108, a sequencing library enriched by direct targeted sequencing is sequenced for a plurality of amounts of a critical parameter (such as different amounts of a sequencing library, different amounts of a capture probe library, or different numbers of amplification cycles). At step 110, the average cluster density and the average cluster intensity are determined for each amount of the critical parameter. At step 112, a plurality of amounts of the critical parameter that provide a desired average cluster density (i.e., an average cluster density that overlaps with a variance of the highest average cluster density, or a cluster density variance that overlaps with the variance of the highest average cluster density is selected, wherein the highest average cluster density and the average cluster densities provided by the plurality of selected amounts of the critical parameter are within a predetermined cluster density range). At step 114, from the plurality of amounts of the critical parameter selected in step 112, the amount of the critical parameter that provides the highest average cluster intensity is selected.

### Selecting a Critical Parameter Based on an Average Cluster Density and an Average Sequencing Quality Metric

In some embodiments, one more critical parameters are selected based on cluster density and an average sequencing quality metric. A sequencing quality metric is a quantitative measurement for evaluating the quality of sequencing data, such as a sequencing quality score (for example a percent Q30 quality score) or a percentage of clusters passing filter. A plurality of amounts of the critical parameter are selected based on cluster density; and from the plurality of amounts of the critical parameter selected based on cluster density, an amount of a critical parameter is selected based on the average sequencing quality metric. For example, in some embodiments, there is a method for selecting an amount of a critical parameter (such as an amount of a sequencing library, and amount of a capture probe library, or a number of amplification cycles) for direct targeted sequencing, comprising sequencing a sequencing library enriched by direct targeted sequencing at a plurality of different amounts of the critical parameter to determine an average cluster density and an sequencing quality score after a predetermined number of sequencing cycles for each critical parameter amount; selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range; and selecting an the amount of the sequencing library that provides the highest average sequencing quality metric from the plurality of selected amounts of the sequencing library that provide an average cluster density that overlaps with a variance of the highest average cluster density or a cluster density variance that overlaps with the variance of the highest average cluster density.

For each amount of the critical parameter, an average cluster density is determined. The highest average cluster density within the predetermined cluster density range is then determined. The highest average cluster density is associated with a variance. From those amounts of the critical parameter that provide an average cluster density that overlaps with a variance of the highest average cluster density, or provide a cluster density variance that overlaps with the variance of the highest average cluster density, an amount of the critical parameter can be selected that provides the highest average sequencing quality metric. In some embodiments, the variance is a statistical variance (e.g., a standard deviation, interquartile range, a statistical dispersion, or other statistical variance). The statistical variance can be determined, for example, based on the cluster density variation on the surface for the amount of the critical parameter. For example, some surfaces include a plurality of tiles, and a cluster density is determined for each tile. A statistical variance can be determined for the amount of the critical parameter that provided the highest average cluster density from the cluster density variance of the tiles. In some embodiments, the variance is percentage of (e.g., within 5% or less, within 10% or less, within 15% or less, or within 20% or less) the determined highest average cluster density. In some embodiments, the variance is a percentile (for example, 70th percentile or above, 80th percentile or above, or 90th percentile or above) for the average cluster densities in the pluralities of amounts of the critical parameters. In some embodiments, the selected plurality of amounts of the critical parameter provide an average cluster density that overlaps with the variance of the highest average cluster density (that is, the average cluster density provided by each of the selected amounts of the critical parameter are within the variance (e.g., statistical variance, percentage of, or percentile) of the highest average cluster density). In some embodiments, the selected plurality of amounts of the critical parameter have a variance (e.g., a statistical variance or a percentage of) associated with the determined average cluster density, and that variance overlaps the variance associated with the highest average cluster density. The variances need not fully overlap as long as some portion of the variances overlap. The selected amounts of the critical parameter, each provide an average cluster density (including the highest average cluster density) within the predetermined cluster density range.

From the plurality of amounts of the critical parameter that provide an average cluster density that overlaps with a variance of the highest average cluster density or a cluster density variance that overlaps with the variance of the highest average cluster density (as long as the average cluster density for the plurality of amounts of the critical parameter is within the predetermined cluster density range), an amount of the critical parameter that provides the highest average sequencing quality metric is selected. The average sequencing quality metric is determined for at least the amounts of the critical parameter in the plurality of amounts of the critical parameter that provide an average cluster density that overlaps with a variance of the highest average cluster density or a cluster density variance that overlaps with the variance of the highest average cluster density, although in some embodiments the average sequencing quality metric is determined for each amount of the critical parameter for which an average cluster density was determined.

FIG. 1C illustrates a method for selecting an amount of a critical parameter for direct targeted sequencing based on an average cluster density and an average sequencing quality metric after a predetermined number of sequencing cycles. At step 116, a sequencing library enriched by direct targeted sequencing is sequenced for a plurality of amounts of a critical parameter (such as different amounts of a sequencing library, different amounts of a capture probe library, or different numbers of amplification cycles). At step 118, the average cluster density and the average sequencing quality metric are determined for each amount of the critical parameter. At step 120, a plurality of amounts of the critical parameter that provide a desired average cluster density (i.e., an average cluster density that overlaps with a variance of the highest average cluster density, or a cluster density variance that overlaps with the variance of the highest average cluster density is selected, wherein the highest average cluster density and the average cluster densities provided by the plurality of selected amounts of the critical parameter are within a predetermined cluster density range). At step 122, from the plurality of amounts of the critical parameter selected in step 120, the amount of the critical parameter that provides the highest average sequencing quality metric is selected.

### Selecting a Critical Parameter Based on an Average Cluster Density, an Average Sequencing Quality Metric, and an Average Cluster Intensity

In some embodiments, one more critical parameters are selected based on cluster density, an average sequencing quality metric, and an average cluster intensity. First, a plurality of amounts of the critical parameter is selected based on cluster density. From the plurality of amounts of the critical parameter selected based on cluster density, a plurality of amounts of the critical parameter is selected based on the average sequencing quality metric. From the plurality of amount of the critical parameter selected based on the average sequencing quality metric, a final amount of the critical parameter is based on the highest average cluster intensity. For example, in some embodiments, there is a method for selecting an amount of a critical parameter (such as an amount of a sequencing library, and amount of a capture probe library, or a number of amplification cycles) for direct targeted sequencing, comprising sequencing a sequencing library enriched by direct targeted sequencing at a plurality of different amounts of the critical parameter to determine an average cluster density, an average sequencing quality metric, and an average cluster intensity for each critical parameter amount after a predetermined number of sequencing cycles; selecting a plurality of amounts of the critical parameter that provide an average cluster density that overlaps with a variance of the highest average cluster density, or a cluster density variance that overlaps with the variance of the highest average cluster density, wherein the highest average cluster density and the average cluster densities provided by the plurality of selected amounts of the critical parameter are within a predetermined cluster density range; selecting a plurality of amounts of the critical parameter that provide an average sequencing quality metric that overlaps with a variance of the highest average sequencing quality metric, or a sequencing quality metric variance that overlaps with the variance of the highest average sequencing quality metric, from the plurality of selected amounts of the critical that provide an average cluster density that overlaps with a variance of the highest average cluster density or a cluster density variance that overlaps with the variance of the highest average cluster density; and selecting the amount of the critical that provides the highest average cluster intensity from the plurality of selected amounts of the critical parameter that provide an average sequencing quality metric that overlaps with a variance of the highest average sequencing quality metric, or a sequencing quality metric variance that overlaps with the variance of the highest average sequencing quality metric.

For each amount of the critical parameter, an average cluster density is determined. The highest average cluster density within the predetermined cluster density range is then determined. The highest average cluster density is associated with a variance. From those amounts of the critical parameter that provide an average cluster density that overlaps with a variance of the highest average cluster density, or provide a cluster density variance that overlaps with the variance of the highest average cluster density, an amount of the critical parameter can be selected that provides the highest average sequencing quality metric. In some embodiments, the variance is a statistical variance (e.g., a standard deviation, interquartile range, a statistical dispersion, or other statistical variance). The statistical variance can be determined, for example, based on the cluster density variation on the surface for the amount of the critical parameter. For example, some surfaces include a plurality of tiles, and a cluster density is determined for each tile. A statistical variance can be determined for the amount of the critical parameter that provided the highest average cluster density from the cluster density variance of the tiles. In some embodiments, the variance is percentage of (e.g., within 5% or less, within 10% or less, within 15% or less, or within 20% or less) the determined highest average cluster density. In some embodiments, the variance is a percentile (for example, 70th percentile or above, 80th percentile or above, or 90th percentile or above) for the average cluster densities in the pluralities of amounts of the critical parameters. In some embodiments, the selected plurality of amounts of the critical parameter provide an average cluster density that overlaps with the variance of the highest average cluster density (that is, the average cluster density provided by each of the selected amounts of the critical parameter are within the variance (e.g., statistical variance, percentage of, or percentile) of the highest average cluster density). In some embodiments, the selected plurality of amounts of the critical parameter have a variance (e.g., a statistical variance or a percentage of) associated with the determined average cluster density, and that variance overlaps the variance associated with the highest average cluster density. The variances need not fully overlap as long as some portion of the variances overlap. The selected amounts of the critical parameter, each provide an average cluster density (including the highest average cluster density) within the predetermined cluster density range.

From the plurality of amounts of the critical parameter that provide an average cluster density that overlaps with a variance of the highest average cluster density or a cluster density variance that overlaps with the variance of the highest average cluster density (as long as the average cluster density for the plurality of amounts of the critical parameter is within the predetermined cluster density range), an amount of the critical parameter is selected that provides the an average sequencing quality metric that overlaps with a variance of the highest average sequencing quality metric, or a sequencing quality metric variance that overlaps with the variance of the highest average sequencing quality metric. The average sequencing quality metric is determined for at least the amounts of the critical parameter in the plurality of amounts of the critical parameter that provide an average cluster density that overlaps with a variance of the highest average cluster density or a cluster density variance that overlaps with the variance of the highest average cluster density, although in some embodiments the average sequencing quality metric is determined for each amount of the critical parameter for which an average cluster density was determined.

The average sequencing quality metric is the average based on one or more tiles of the sequencing surface. If the surface only includes a single tile, the average sequencing quality metric is the sequencing quality metric for that tile. From those amounts of the critical parameter that provide an average cluster density that of the critical parameter that provide an average cluster density that overlaps with a variance of the highest average cluster density, or a cluster density variance that overlaps with the variance of the highest average cluster density, an average sequencing quality metric is determined. From the determined average sequencing quality metrics, the highest average sequencing quality metric can be determined, along with a variance associated with the highest average sequencing quality metric. In some embodiments, a variance of the sequencing quality metric is determined for the critical parameters for which an average an average sequencing quality metric is determined. In some embodiments, the variance is a statistical variance (e.g., a standard deviation, interquartile range, a statistical dispersion, or other statistical variance). The statistical variance can be determined, for example, based on the cluster density variation on the surface for the amount of the critical parameter. For example, some surfaces include a plurality of tiles, and a cluster density is determined for each tile. A statistical variance can be determined for the amount of the critical parameter that provided the highest average cluster density from the cluster density variance of the tiles. In some embodiments, the variance is percentage of (e.g., within 5% or less, within 10% or less, within 15% or less, or within 20% or less) the determined highest average cluster density. In some embodiments, the variance is a percentile (for example, 70th percentile or above, 80th percentile or above, or 90th percentile or above) for the average cluster densities in the pluralities of amounts of the critical parameters. In some embodiments, the selected plurality of amounts of the critical parameter provide an average sequencing quality metric that overlaps with the variance of the highest average sequencing quality metric (that is, the average sequencing quality metric provided by each of the selected amounts of the critical parameter are within the variance (e.g., statistical variance, percentage of, or percentile) of the highest average sequencing quality metric). In some embodiments, the selected plurality of amounts of the critical parameter have a variance (e.g., a statistical variance or a percentage of) associated with the determined average sequencing quality metric, and that variance overlaps with the variance associated with the highest average sequencing quality metric. The variances need not fully overlap as long as some portion of the variances overlap.

The sequencing quality metric can be, for example, a percent sequencing quality score (for example, a percent Q10 quality score, a percent Q20 quality score, or a percent Q30 quality score) or a percentage of clusters passing filter

From the plurality of amounts of the critical parameter that provide an average sequencing quality metric that overlaps with a variance of the highest average sequencing quality metric, or a sequencing quality metric variance that overlaps with the variance of the highest average sequencing quality metric, an amount of the critical parameter that provides the highest average cluster intensity is selected. The average cluster intensity is determined for at least those amounts of the critical parameter that provide an average sequencing quality metric that overlaps with a variance of the highest average sequencing quality metric, or a sequencing quality metric variance that overlaps with the variance of the highest average sequencing quality metric.

FIG. 1D illustrates a method for selecting an amount of a critical parameter for direct targeted sequencing based on an average cluster density, an average sequencing quality metric, and an average cluster intensity after a predetermined number of sequencing cycles. At step 124, a sequencing library enriched by direct targeted sequencing is sequenced for a plurality of amounts of a critical parameter (such as different amounts of a sequencing library, different amounts of a capture probe library, or different numbers of amplification cycles). At step 126, the average cluster density, the average sequencing quality metric, and the average cluster intensity are determined for each amount of the critical parameter. At step 128, a plurality of amounts of the critical parameter that provide a desired average cluster density (i.e., an average cluster density that overlaps with a variance of the highest average cluster density, or a cluster density variance that overlaps with the variance of the highest average cluster density, wherein the highest average cluster density and the average cluster densities provided by the plurality of selected amounts of the critical parameter are within a predetermined cluster density range) is selected. At step 130, from the plurality of amounts of the critical parameter selected in step 128, a plurality of amounts of the critical parameter that provides a desired average sequencing quality metric (i.e., an average sequencing quality metric that overlaps with a variance of the highest average sequencing quality metric, or a sequencing quality metric variance that overlaps with the variance of the highest average sequencing quality metric) is selected. At step 132, from the plurality of amounts of the critical parameter selected in step 130, the amount of the critical parameter that provides the highest average cluster intensity is selected.

### Selection of an Amount for Multiple Critical Parameters

In some embodiments, amounts for multiple (e.g., two or three) critical parameters are selected. The amounts for multiple critical parameters can be selected sequentially (i.e., selecting an amount of the first critical parameter, selecting an amount of the second critical parameter using the selected amount of the first critical parameter, and, optionally, selecting the amount of a third critical parameter using the selected amount of the first critical parameter and the selected amount of the second critical parameter) or simultaneously (i.e., the first critical parameter, the second critical parameter, and optionally the third critical parameter are selected simultaneously using different combinations of amounts of the critical parameters using a multi-parameter matrix.

In some embodiments, an amount of sequencing library and an amount of capture probe library are selected. In some embodiments, an amount of sequencing library and a number of amplification cycles are selected. In some embodiments, an amount of capture probe library and a number of amplification cycles are selected. In some embodiments, an amount of sequencing library, an amount of capture probe library, and a number of amplification cycles are selected.

### Sequential Selection of Multiple Critical Parameters

In some embodiments, the amounts of multiple critical parameters are selected sequentially. In some embodiments, the amount of the first critical parameter is selected by sequencing a sequencing library enriched by direct targeted sequencing at a plurality of different amounts of the first critical parameter and holding the amounts of the remaining critical parameters (e.g., the second critical parameter and the third critical parameter) constant. Once the amount of the first critical parameter is selected, the amount of the second critical parameter is selected by sequencing a sequencing library enriched by direct targeted sequencing at a plurality of different amounts of the second critical parameter and holding the amounts of the remaining critical parameters (e.g., the first critical parameter and the third critical parameter) constant, wherein the amount of the first critical parameter is the selected amount of the first critical parameter. Optionally, once the amount of the second critical parameter is selected, the amount of the third critical parameter is selected by sequencing a sequencing library enriched by direct targeted sequencing at a plurality of different amounts of the third critical parameter and holding the amounts of the remaining critical parameters (e.g., the first critical parameter and the second critical parameter) constant, wherein the amount of the first critical parameter is the selected amount of the first critical parameter and the amount of the second critical parameter is the selected amount of the second critical parameter.

In some embodiments, the amounts of the critical parameters are determined iteratively. For example, an amount of a first critical parameter can be selected holding a second critical parameter constant; then an amount of the second critical parameter can be selected holding the first critical parameter at the initially selected amount; and then the amount of the first critical parameter can be re-selected by sequencing the sequencing library enriched by direct targeted sequencing at a plurality of different amounts of the first critical parameter and holding the amounts of the second critical parameter constant at the selected amount of the second critical parameter.

In some embodiments, the amount of the sequencing library and the amount of the capture probe library are sequentially determined. For example, the amount of sequencing library is first selected by sequencing a sequencing library enriched by direct targeted sequencing at a plurality of different amounts of the sequencing library and holding the amount of the capture probe library and the number of amplification cycles constant. The different amounts of the sequencing library are from within a predetermined range. Next the amount of capture probe library is selected by sequencing the sequencing library enriched by direct targeted sequencing at a plurality of different amounts of the capture probe library and holding the amount of the sequencing library and the number of amplification cycles constant, wherein the amount of the sequencing library is the selected amount of the sequencing library. In another example, the amount of capture probe library is first selected by sequencing a sequencing library enriched by direct targeted sequencing at a plurality of different amounts of the capture probe library and holding the amount of the sequencing library and the number of amplification cycles constant. The different amounts of the capture probe library are from within a predetermined range. Next the amount of sequencing library is selected by sequencing the sequencing library enriched by direct targeted sequencing at a plurality of different amounts of the sequencing library and holding the amount of the capture probe library and the number of amplification cycles constant, wherein the amount of the capture probe library is the selected amount of the capture probe library.

In some embodiments, the amount of the sequencing library and the number of amplification cycles are sequentially determined. For example, the amount of sequencing library is first selected by sequencing a sequencing library enriched by direct targeted sequencing at a plurality of different amounts of the sequencing library and holding the amount of the capture probe library and the number of amplification cycles constant. The different amounts of the sequencing library are from within a predetermined range. Next the number of amplification cycles is selected by sequencing the sequencing library enriched by direct targeted sequencing at a plurality of different numbers of amplification cycles and holding the amount of the sequencing library and the amount of the capture probe library constant, wherein the amount of the sequencing library is the selected amount of the sequencing library. In another example, the number of amplification cycles is first selected by sequencing a sequencing library enriched by direct targeted sequencing at a plurality of different numbers of amplification cycles and holding the amount of the sequencing library and the amount of capture probe library constant. The different numbers of amplification cycles are from within a predetermined range. Next the amount of sequencing library is selected by sequencing the sequencing library enriched by direct targeted sequencing at a plurality of different amounts of the sequencing library and holding the amount of the capture probe library and the number of amplification cycles constant, wherein the number of amplification cycles is the selected number of amplification cycles.

In some embodiments, the amount of the capture probe library and the number of amplification cycles are sequentially determined. For example, the amount of capture probe library is first selected by sequencing a sequencing library enriched by direct targeted sequencing at a plurality of different amounts of the capture probe library and holding the amount of the sequencing library and the number of amplification cycles constant. The different amounts of the capture probe library are from within a predetermined range. Next the number of amplification cycles is selected by sequencing the sequencing library enriched by direct targeted sequencing at a plurality of different numbers of amplification cycles and holding the amount of the sequencing library and the amount of the capture probe library constant, wherein the amount of the capture probe library is the selected amount of the sequencing library. In another example, the number of amplification cycles is first selected by sequencing a sequencing library enriched by direct targeted sequencing at a plurality of different numbers of amplification cycles and holding the amount of the sequencing library and the amount of capture probe library constant. The different numbers of amplification cycles are from within a predetermined range. Next the amount of capture probe library is selected by sequencing the sequencing library enriched by direct targeted sequencing at a plurality of different amounts of the capture library and holding the amount of the sequencing library and the number of amplification cycles constant, wherein the number of amplification cycles is the selected number of amplification cycles.

In some embodiments, the amount of the sequencing library, the amount of the capture probe library, and the number of amplification cycles are sequentially determined. For example, the amount of sequencing library can be first selected by sequencing a sequencing library enriched by direct targeted sequencing at a plurality of different amounts of the sequencing library and holding the amount of the capture probe library and the number of amplification cycles constant. The different amounts of the sequencing library are from within a predetermined range. Next the amount of capture probe library is selected by sequencing the sequencing library enriched by direct targeted sequencing at a plurality of different amounts of the capture probe library and holding the amount of the sequencing library and the number of amplification cycles constant, wherein the amount of the sequencing library is the selected amount of the sequencing library. Finally, the number of amplification cycles is selected by sequencing the sequencing library enriched by direct targeted sequencing at a plurality of different numbers of amplification cycles and holding the amount of the sequencing library and the amount of the capture probe library constant, wherein the amount of the sequencing library is the selected amount of the sequencing library and the amount of the capture probe library is the selected amount of the capture probe library.

In another example, the amount of sequencing library can be first selected by sequencing a sequencing library enriched by direct targeted sequencing at a plurality of different amounts of the sequencing library and holding the amount of the capture probe library and the number of amplification cycles constant. The different amounts of the sequencing library are from within a predetermined range. Next the number of amplification cycles is selected by sequencing the sequencing library enriched by direct targeted sequencing at a plurality of different number of amplification cycles and holding the amount of the sequencing library and the amount of the capture probe library constant, wherein the amount of the sequencing library is the selected amount of the sequencing library. Finally, the amount of the capture probe library is selected by sequencing the sequencing library enriched by direct targeted sequencing at a plurality of different amounts of the capture probe library and holding the amount of the sequencing library and the number of amplification cycles constant, wherein the amount of the sequencing library is the selected amount of the sequencing library and the number of amplification cycles is the selected number of amplification cycles.

In another example, the amount of capture probe library can be first selected by sequencing a sequencing library enriched by direct targeted sequencing at a plurality of different amounts of the capture probe library and holding the amount of the sequencing library and the number of amplification cycles constant. The different amounts of the capture probe library are from within a predetermined range. Next the amount of sequencing library is selected by sequencing the sequencing library enriched by direct targeted sequencing at a plurality of different amounts of the sequencing library and holding the amount of the capture probe library and the number of amplification cycles constant, wherein the amount of the capture probe library is the selected amount of the capture probe library. Finally, the number of amplification cycles is selected by sequencing the sequencing library enriched by direct targeted sequencing at a plurality of different numbers of amplification cycles and holding the amount of the sequencing library and the amount of the capture probe library constant, wherein the amount of the sequencing library is the selected amount of the sequencing library and the amount of the capture probe library is the selected amount of the capture probe library.

In another example, the amount of capture probe library can be first selected by sequencing a sequencing library enriched by direct targeted sequencing at a plurality of different amounts of the capture probe library and holding the amount of the sequencing library and the number of amplification cycles constant. The different amounts of the capture probe library are from within a predetermined range. Next the number of amplification cycles is selected by sequencing the sequencing library enriched by direct targeted sequencing at a plurality of different number of amplification cycles and holding the amount of the sequencing library and the amount of the sequencing library constant, wherein the amount of the sequencing library is the selected amount of the sequencing library. The different numbers of amplification cycle are from within a predetermined range. Finally, the amount of the sequencing library is selected by sequencing the sequencing library enriched by direct targeted sequencing at a plurality of different amounts of the sequencing library and holding the amount of the capture probe library and the number of amplification cycles constant, wherein the amount of the capture probe library is the selected amount of the capture probe library and the number of amplification cycles is the selected number of amplification cycles. The different amounts of the sequencing library are from within a predetermined range.

In another example, the number of amplification cycles can be first selected by sequencing a sequencing library enriched by direct targeted sequencing at a plurality of different numbers of amplification cycles and holding the amount of the sequencing library and the amount of the capture probe library constant. The different numbers of amplification cycles are from within a predetermined range. Next the amount of sequencing library is selected by sequencing the sequencing library enriched by direct targeted sequencing at a plurality of different amounts of the sequencing library and holding the amount of the capture probe library and the number of amplification cycles constant, wherein the number of amplification cycles is the selected number of amplification cycles. The different amounts of the sequencing library are from within a predetermined range. Finally, the amount of the capture probe library is selected by sequencing the sequencing library enriched by direct targeted sequencing at a plurality of different amounts of the capture probe library and holding the amount of the sequencing library and the number of amplification cycles constant, wherein the amount of the sequencing library is the selected amount of the sequencing library and the number of amplification cycles is the selected number of amplification cycles. The different amounts of the capture probe library can be from within a predetermined range.

In another example, the number of amplification cycles can be first selected by sequencing a sequencing library enriched by direct targeted sequencing at a plurality of different numbers of amplification cycles and holding the amount of the sequencing library and the amount of capture probe library constant. The different numbers of amplification cycles are from within a predetermined range. Next the amount of capture probe library is selected by sequencing the sequencing library enriched by direct targeted sequencing at a plurality of different amounts of the capture probe library and holding the amount of the sequencing library and the number of amplification cycles constant, wherein the number of amplification cycles is the selected number of amplification cycles. The different amounts of the capture probe library are from within a predetermined range. Finally, the amount of the sequencing library is selected by sequencing the sequencing library enriched by direct targeted sequencing at a plurality of different amounts of the sequencing library and holding the amount of the capture probe library and the number of amplification cycles constant, wherein the amount of the capture probe library is the selected amount of the sequencing library and the number of amplification cycles is the selected number of amplification cycles. The different amounts of the sequencing library are from within a predetermined range.

### Simultaneous Selection of Amounts of Multiple Critical Parameters

In some embodiments, the amounts of multiple critical parameters (for example two or three different critical parameters) are selected simultaneously. This can be done by sequencing a sequencing a sequencing library enriched by direct targeted sequencing at a plurality of different amounts of the first critical parameter and a plurality of different amounts of the second critical parameter (and, optionally, a plurality of different amounts of the third critical parameter). For example, in some embodiments, there is provided a method for selecting an amount of a first critical parameter and an amount of a second critical parameter (and, optionally, an amount of a third critical parameter) for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different combinations of amounts of the first critical parameter and amounts of the second critical parameter (and the optional third critical parameter); and (i) selecting the combination of the amount of the first critical parameter and the amount of the second critical parameter (and the optional third critical parameter) that provides: (1) the highest average cluster density, (2) an average cluster density that overlaps with a variance of the highest average cluster density, or (3) a cluster density variance that overlaps with the variance of the highest average cluster density, wherein the highest average cluster density and the average cluster density provided by the selected amount of the sequencing library are within a predetermined cluster density range.

The plurality of different combinations of the amount of the first critical parameter and the second critical parameter (and the optional third critical parameter) can be selected based on a two-dimensional (or three-dimensional) multi-parameter matrix. For example, each amount within a plurality of amounts of the first critical parameter is combined with an amount of the second critical parameter from the plurality of amounts of the second critical parameter to form a plurality of combinations. For example, if a plurality of amounts of the first critical parameter includes 10 different amounts and a plurality of amounts of the second critical parameter includes 5 different amounts, steps (a)-(g) can be repeated for up to 50 different combinations.

In some embodiments, there is provided a method for selecting an amount of a first critical parameter and an amount of a second critical parameter (and, optionally, an amount of a third critical parameter) for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average sequencing quality metric after a predetermined number of sequencing cycles; (h) repeating steps (a)-(g) at a plurality of different combinations of amounts of the first critical parameter and amounts of the second critical parameter (and the optional third critical parameter); and (i) selecting the combination of the amount of the first critical parameter and the amount of the second critical parameter (and the optional third critical parameter) that provides an average cluster density that overlaps with a variance of the highest average cluster density, or a cluster density variance that overlaps with the variance of the highest average cluster density, wherein the highest average cluster density and the average cluster density provided by the selected amount of the sequencing library are within a predetermined cluster density range; and (j) selecting the combination that provides the highest average sequencing quality metric from the plurality of selected combinations that provide an average cluster density that overlaps with a variance of the highest average cluster density or a cluster density variance that overlaps with the variance of the highest average cluster density.

In some embodiments, there is provided a method for selecting an amount of a first critical parameter and an amount of a second critical parameter (and, optionally, an amount of a third critical parameter) for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density, an average sequencing quality metric, and an average cluster intensity after a predetermined number of sequencing cycles; (h) repeating steps (a)-(g) at a plurality of different combinations of amounts of the first critical parameter and amounts of the second critical parameter (and the optional third critical parameter); and (i) selecting the combination of the amount of the first critical parameter and the amount of the second critical parameter (and the optional third critical parameter) that provides an average cluster density that overlaps with a variance of the highest average cluster density, or a cluster density variance that overlaps with the variance of the highest average cluster density, wherein the highest average cluster density and the average cluster density provided by the selected amount of the sequencing library are within a predetermined cluster density range; (j) selecting a plurality of combinations that provide an average sequencing quality metric that overlaps with a variance of the highest average sequencing quality metric, or a sequencing quality metric variance that overlaps with the variance of the highest average sequencing quality metric, from the plurality of combinations that provide an average cluster density that overlaps with a variance of the highest average cluster density or a cluster density variance that overlaps with the variance of the highest average cluster density; and (k) selecting the combination that provides the highest average cluster intensity from the plurality of selected combinations that provide an average sequencing quality metric that overlaps with a variance of the highest average sequencing quality metric, or a sequencing quality metric variance that overlaps with the variance of the highest average sequencing quality metric

In some embodiments, there is provided a method for selecting an amount of a first critical parameter and an amount of a second critical parameter (and, optionally, an amount of a third critical parameter) for direct targeted sequencing, comprising: (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average cluster intensity after a predetermined number of sequencing cycles; (h) repeating steps (a)-(g) at a plurality of different combinations of amounts of the first critical parameter and amounts of the second critical parameter (and the optional third critical parameter); and (i) selecting the combination of the amount of the first critical parameter and the amount of the second critical parameter (and optionally the amount of the third critical parameter) that provides an average cluster density that overlaps with a variance of the highest average cluster density, or a cluster density variance that overlaps with the variance of the highest average cluster density, wherein the highest average cluster density and the average cluster density provided by the selected amount of the sequencing library are within a predetermined cluster density range; and (j) selecting the combination that provides the highest average cluster intensity from the plurality of selected combinations that provide an average cluster density that overlaps with a variance of the highest average cluster density or a cluster density variance that overlaps with the variance of the highest average cluster density.

In some embodiments, the amount of the sequencing library and the amount of the capture probe library are simultaneously selected (that is, by repeating the direct targeted sequencing steps using a plurality of different combinations of amounts of the sequencing library and amounts of the capture probe library). In some embodiments, the amount of the sequencing library and the number of amplification cycles are simultaneously selected. In some embodiments, the amount of the capture probe library and the number of amplification cycles are simultaneously selected. In some embodiments, the amount of the capture probe library, the amount of the sequencing library, and the number of amplification cycles are simultaneously selected.

In some embodiments, the amounts of three critical parameters are selected by a combination of sequential selection and simultaneous selection. For example, in some embodiments a first critical parameter is selected by sequencing a sequencing library enriched by direct targeted sequencing at a plurality of different amounts of the first critical parameter and holding the amount of the second critical parameter and the amount of the third critical parameter constant, and then selecting the amount of the second critical parameter and the amount of the third critical parameter simultaneously by sequencing the sequencing library enriched by direct targeted sequencing at a plurality of different combinations of an amount of the second critical parameter and the third critical parameter, wherein the amount of the first critical parameter is held constant at the selected amount of the first critical parameter. In another example, in some embodiments, an amount of a first critical parameter and an amount of a second critical parameter is simultaneously selected by sequencing the sequencing library enriched by direct targeted sequencing at a plurality of different combinations of an amount of the first critical parameter and the second critical parameter and holding the third critical parameter constant, and then selecting the third critical parameter by sequencing a sequencing library enriched by direct targeted sequencing at a plurality of different amounts of the third critical parameter and holding the amount of the first critical parameter and the amount of the second critical parameter constant, wherein the amount of the first critical parameter is the selected amount of the first critical parameter and the amount of the second critical parameter is the selected amount of the second critical parameter.

### Critical Parameters

The methods described herein are useful for selecting an amount of one or critical parameters for direct targeted sequencing. The critical parameters include an amount of a sequencing library, an amount of a capture probe library, and a number of amplification cycles. In some embodiments, the method is used to select an amount of one critical parameter. In some embodiments, the method is used to select an amount of two critical parameters. In some embodiments, the method is used to select an amount of three critical parameters. Not all critical parameters are required to be selected using the methods described herein. Amounts of one or more critical parameters can be used for direct targeted sequencing, for example by selecting an amount of the critical parameter based on methods known in the art (for example, sequence manufacturer recommendations).

### Critical Parameter—Sequencing Library

In some embodiments, an amount of the sequencing library is selected for direct targeted sequencing. The sequencing library includes a plurality of nucleic acid molecules, which can be isolated from a sample (for example, a blood, saliva, plasma, or tissue sample). The sequencing library includes a region of interest (that is, the portion of the genetic information enriched by the capture probes in the direct targeted sequencing methods).

The present invention provides methods for enhancing direct targeted sequencing by titrating the amount of sequencing library. In some embodiments, the amount of sequencing library selected by the method described herein is in excess of the amount used in previous direct targeted sequencing efforts. Prior to the present invention, it was reported that “an increase in the library concentration did not lead to a significant increase in on-target sequence.” (Hopmans et al., “A programmable method for massively parallel targeted sequencing.” Nucleic Acids Res. 42(10):e88 (2014)). Specifically Hopmans et al. showed that, “after 20 h of hybridization with 500 ng of sequencing library, ˜4.9% of all potential targets within the sequencing library were captured for sequencing” and it was concluded that, “therefore, library fragments are available in excess for optimal capture and do not require exact titration.” (Hopmans et al., “A programmable method for massively parallel targeted sequencing.” Nucleic Acids Res. 42(10):e88 (2014)).

By contrast, the present invention identifies the amount of sequencing library as a critical parameter for the direct targeted sequencing method. Surprisingly, it was further found that a desirable amount of the sequencing library can be identified by titrating the amount of sequencing library, using increasing amounts of sequencing library that are 200× to 2000× greater than the amount previously used (compare to amounts used in Myllykangas et al. “Efficient targeted resequencing of human germline and cancer genomes by oligonucleotide-selective sequencing.” Nat Biotechnol. 29(11):1024-7 (2011); and Hopmans et al., “A programmable method for massively parallel targeted sequencing.” Nucleic Acids Res. 42(10):e88 (2014)).

In some embodiments, an amount of a sequencing library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different amounts of the sequencing library; and (i) selecting an amount of the sequencing library that provides: (1) the highest average cluster density, (2) an average cluster density that overlaps with a variance of the highest average cluster density, or (3) a cluster density variance that overlaps with the variance of the highest average cluster density, wherein the highest average cluster density and the average cluster density provided by the selected amount of the sequencing library are within a predetermined cluster density range. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the sequencing library is a predetermined percentage of the average cluster density provided by the selected amount of the sequencing library. In some embodiments, the cluster density variance provided by the selected amount of the sequencing library is a predetermined statistical variance of the cluster density provided by the selected amount of the sequencing library.

In some embodiments, an amount of a sequencing library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different amounts of the sequencing library; and (i) selecting the amount of the sequencing library that provides the highest average cluster density, wherein the highest average cluster density is within a predetermined cluster density range.

In some embodiments, an amount of a sequencing library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average sequencing quality metric after a predetermined number of sequencing cycles; (h) repeating steps (a)-(g) at a plurality of different amounts of the sequencing library; (i) selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within the predetermined cluster density range; and (j) selecting the amount of the sequencing library that provides the highest average sequencing quality metric from the plurality of selected amounts of the sequencing library that provide an average cluster density that overlaps with a variance of the highest average cluster density or a cluster density variance that overlaps with the variance of the highest average cluster density. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the sequencing library is a predetermined percentage of the average cluster density provided by the selected amount of the sequencing library. In some embodiments, the cluster density variance provided by the selected amount of the sequencing library is a predetermined statistical variance of the cluster density provided by the selected amount of the sequencing library.

In some embodiments, an amount of a sequencing library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density, an average sequencing quality metric, and an average cluster intensity after a predetermined number of sequencing cycles; (h) repeating steps (a)-(g) at a plurality of different amounts of the sequencing library; (i) selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within the predetermined cluster density range; (j) selecting a plurality of amounts of the sequencing library that provide an average sequencing quality metric that overlaps with a variance of the highest average sequencing quality metric, or a sequencing quality metric variance that overlaps with the variance of the highest average sequencing quality metric, from the plurality of selected amounts of the sequencing library that provide an average cluster density that overlaps with a variance of the highest average cluster density or a cluster density variance that overlaps with the variance of the highest average cluster density; and (k) selecting the amount of the sequencing library that provides the highest average cluster intensity from the plurality of selected amounts of the sequencing library that provide an average sequencing quality metric that overlaps with a variance of the highest average sequencing quality metric, or a sequencing quality metric variance that overlaps with the variance of the highest average sequencing quality metric. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the sequencing library is a predetermined percentage of the average cluster density provided by the selected amount of the sequencing library. In some embodiments, the cluster density variance provided by the selected amount of the sequencing library is a predetermined statistical variance of the cluster density provided by the selected amount of the sequencing library.

In some embodiments, an amount of a sequencing library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average cluster intensity after a predetermined number of sequencing cycles; (h) repeating steps (a)-(g) at a plurality of different amounts of the sequencing library; (i) selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within the predetermined cluster density range; and (j) selecting an the amount of the sequencing library that provides the highest average cluster intensity from plurality of selected amounts of the sequencing library that provide an average cluster density that overlaps with a variance of the highest average cluster density or a cluster density variance that overlaps with the variance of the highest average cluster density. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the sequencing library is a predetermined percentage of the average cluster density provided by the selected amount of the sequencing library. In some embodiments, the cluster density variance provided by the selected amount of the sequencing library is a predetermined statistical variance of the cluster density provided by the selected amount of the sequencing library.

Selection of the amount of the sequencing library can include repeating steps (a)-(g) at a plurality of amounts for one or more additional critical parameters (such as a plurality of amounts of the capture probe library or a plurality of numbers of amplification cycles), which can be selected sequentially or simultaneously.

The plurality of different amounts of the sequencing library can include 2 or more different amounts, 3 or more different amounts, 5 or more different amounts, 10 or more different amounts, 25 or more different amounts, or 50 or more different amounts. In some embodiments, the different amounts are within a predetermined range. In some embodiments, the different amounts are evenly spaced or approximately evenly spaced within the range.

In some embodiments, the predetermined range for the amount of the sequencing library is or is set within about 50 μg to about 500 μg (for example, about 75 μg to about 350 μg, about 100 μg to about 250 μg, about 125 μg to about 175 μg, or about 100 μg). In some embodiments, the amount of sequencing library is about 50 μg or more (such as about 75 μg or more, about 100 μg or more, about 125 μg or more, about 150 μg or more, or about 200 μg or more). In some embodiments, the amount of the sequencing library is about 500 μg or less (such as about 400 μg or less, about 350 μg or less, about 300 μg or less, about 250 μg or less, about 200 μg or less, or about 175 μg or less).

In some embodiments, the predetermined range for the amount of the sequencing library is or is set within a concentration of about 1 μM to about 50 μM (for example, about 1 μM to about 5 μM, about 5 μM to about 10 μM, about 10 μM to about 20 μM, or about 20 μM to about 50 μM). In some embodiments, the amount of sequencing library is about 1 μM or more (such as about 2 μM or more, about 2 μM or more, about 3 μM or more, about 5 μM or more, about 7 μM or more, or about 10 μM or more). In some embodiments, the amount of the sequencing library is about 50 μM or less (such as about 40 μM or less, about 20 μM or less, or about 10 μM or less).

### Critical Parameter—Capture Probe Library

In some embodiments, an amount of the capture probe library is selected for direct targeted sequencing. The capture probe includes a plurality of capture probes that are used to enrich the region of interest in the sequencing library. The capture probes include a first end with a sequence that hybridizes to surface-bound oligonucleotides and as second end that has a portion of the region of interest.

In some embodiments, an amount of a capture probe library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different amounts of the capture probe library; and (i) selecting an amount of the capture probe library that provides: (1) the highest average cluster density, (2) an average cluster density that overlaps with a variance of the highest average cluster density, or (3) a cluster density variance that overlaps with the variance of the highest average cluster density, wherein the highest average cluster density and the average cluster density provided by the selected amount of the capture probe library are within a predetermined cluster density range. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the capture probe library is a predetermined percentage of the average cluster density provided by the selected amount of the capture probe library. In some embodiments, the cluster density variance provided by the selected amount of the capture probe library is a predetermined statistical variance of the cluster density provided by the selected amount of the capture probe library.

In some embodiments, an amount of a capture probe library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different amounts of the capture probe library; and (i) selecting the amount of the sequencing library that provides the highest average cluster density, wherein the highest average cluster density is within a predetermined cluster density range.

In some embodiments, an amount of a capture probe library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average sequencing quality metric after a predetermined number of sequencing cycles; (h) repeating steps (a)-(g) at a plurality of different amounts of the capture probe library; (i) selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and (j) selecting the amount of the capture probe library that provides the highest average sequencing quality metric from the plurality of selected amounts of the capture probe library that provide an average cluster density that overlaps with a variance of the highest average cluster density or a cluster density variance that overlaps with the variance of the highest average cluster density. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the capture probe library is a predetermined percentage of the average cluster density provided by the selected amount of the capture probe library. In some embodiments, the cluster density variance provided by the selected amount of the capture probe library is a predetermined statistical variance of the cluster density provided by the selected amount of the capture probe library.

In some embodiments, an amount of a capture probe library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density, an average sequencing quality metric, and an average cluster intensity after a predetermined number of sequencing cycles; (h) repeating steps (a)-(g) at a plurality of different amounts of the capture probe library; (i) selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; (j) selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture probe library that provide an average cluster density that overlaps with a variance of the highest average cluster density or a cluster density variance that overlaps with the variance of the highest average cluster density; and (k) selecting the amount of the capture probe library that provides the highest average cluster intensity from the plurality of selected amounts of the capture probe library that provide an average sequencing quality metric that overlaps with a variance of the highest average sequencing quality metric, or a sequencing quality metric variance that overlaps with the variance of the highest average sequencing quality metric. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the capture probe library is a predetermined percentage of the average cluster density provided by the selected amount of the capture probe library. In some embodiments, the cluster density variance provided by the selected amount of the capture probe library is a predetermined statistical variance of the cluster density provided by the selected amount of the capture probe library.

In some embodiments, an amount of a capture probe library is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average cluster intensity after a predetermined number of sequencing cycles; (h) repeating steps (a)-(g) at a plurality of different amounts of the capture probe library; (i) selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and (j) selecting an the amount of the capture probe library that provides the highest average cluster intensity from plurality of selected amounts of the capture probe library that provide an average cluster density that overlaps with a variance of the highest average cluster density or a cluster density variance that overlaps with the variance of the highest average cluster density. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the capture probe library is a predetermined percentage of the average cluster density provided by the selected amount of the capture probe library. In some embodiments, the cluster density variance provided by the selected amount of the capture probe library is a predetermined statistical variance of the cluster density provided by the selected amount of the capture probe library.

Selection of the amount of the capture probe library can include repeating steps (a)-(g) at a plurality of amounts for one or more additional critical parameters (such as a plurality of amounts of the sequencing library or a plurality of numbers of amplification cycles), which can be selected sequentially or simultaneously.

The plurality of different amounts of the capture probe library can be 2 or more different amounts, 3 or more different amounts, 5 or more different amounts, 10 or more different amounts, 25 or more different amounts, or 50 or more different amounts. In some embodiments, the different amounts are within a predetermined range. In some embodiments, the different amounts are evenly spaced or approximately evenly spaced within the range. In some embodiments, the different amounts are unevenly spaced within the range.

In some embodiments, the predetermined range for the amount of the capture probe library is or is set within about 10 nM to about 250 nM (such as about 20 nM to about 200 nM, about 30 nM to about 150 nM, about 40 nM to about 100 nM, or about 50 nM to about 65 nM). In some embodiments, the amount of the capture probe library is about 10 nM or more (such as about 20 nM or more, about 30 nM or more, about 40 nM or more, or about 50 nM or more). In some embodiments, the amount of the capture probe library is about 250 nM or less (such as about 200 nM or less, about 150 nM or less, about 100 nM or less, about 75 nM or less, or about 65 nM or less).

In some embodiments, the predetermined range for the amount of the capture probe library is or is set within about 100 nanograms (ng) to about 1000 ng, about 150 ng to about 900 ng, about 250 ng to about 800 ng, about 300 ng to about 700 ng, about 400 ng to about 600 ng, or about 425 ng to about 550 ng). In some embodiments, the amount of the capture probe library is about 100 ng or more (such as about 150 ng or more, about 250 ng or more, about 300 ng or more, about 400 ng or more, or about 425 ng or more. In some embodiments, the amount of the capture probe library is about 1000 ng or less (such as about 900 ng or less, about 800 ng or less, about 700 ng or less, about 600 ng or less, about 550 ng or less, or about 500 ng or less).

### Critical Parameter—Amplification Cycles

In some embodiments, a number of amplification cycles (i.e., bridge amplification cycles) is selected for direct targeted sequencing. The number of amplification cycles impacts the number of copies of amplified surface-bound complements of the nucleic acid molecules. During bridge amplification, the surface-bound complements are amplified, forming additional surface-bound complements or complements of the surface-bound complements during each amplification cycle. Although the methods herein described herein refer to “sequencing the amplified surface-bound complements,” it is understood that this can include sequencing the complements of the surface-bound complements. The number of amplified surface-bound complements also impacts the size of the clusters, as well as the cluster intensity and sequencing quality.

In some embodiments, a number of amplification cycles is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different numbers of amplification cycles; and (i) selecting a number of amplification cycles that provides: (1) the highest average cluster density, (2) an average cluster density that overlaps with a variance of the highest average cluster density, or (3) a cluster density variance that overlaps with the variance of the highest average cluster density, wherein the highest average cluster density and the average cluster density provided by the selected amount of the capture probe library are within a predetermined cluster density range. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected number of amplification cycles is a predetermined percentage of the average cluster density provided by the selected amount of the capture probe library. In some embodiments, the cluster density variance provided by the selected number of amplification cycles is a predetermined statistical variance of the cluster density provided by the selected number of amplification cycles.

In some embodiments, a number of amplification cycles is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density after a predetermined number of sequencing cycles; (h) repeating steps (a)-(g) at a plurality of different numbers of amplification cycles; and (i) selecting the number of amplification cycles that provides the highest average cluster density, wherein the highest average cluster density is within a predetermined cluster density range.

In some embodiments, a number of amplification cycles is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average sequencing quality metric after a predetermined number of sequencing cycles; (h) repeating steps (a)-(g) at a plurality of different numbers of amplification cycles; (i) selecting a plurality of a numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and (j) selecting the number of amplification cycles that provides the highest average sequencing quality metric from the plurality of selected a number of amplification cycles that provide an average cluster density that overlaps with a variance of the highest average cluster density or a cluster density variance that overlaps with the variance of the highest average cluster density. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected number of amplification cycles is a predetermined percentage of the average cluster density provided by the selected number of amplification cycles. In some embodiments, the cluster density variance provided by the selected number of amplification cycles is a predetermined statistical variance of the cluster density provided by the selected number of amplification cycles.

In some embodiments, a number of amplification cycles is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density, an average sequencing quality metric, and an average cluster intensity after a predetermined number of sequencing cycles; (h) repeating steps (a)-(g) at a plurality of different numbers of amplification cycles; (i) selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; (j) selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with a variance of the highest average cluster density or a cluster density variance that overlaps with the variance of the highest average cluster density; and (k) selecting the number of amplification cycles that provides the highest average cluster intensity from the plurality of selected amounts of the capture probe library that provide an average sequencing quality metric that overlaps with a variance of the highest average sequencing quality metric, or a sequencing quality metric variance that overlaps with the variance of the highest average sequencing quality metric. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected amount of the capture probe library is a predetermined percentage of the average cluster density provided by the selected number of amplification cycles. In some embodiments, the cluster density variance provided by the selected number of amplification cycles is a predetermined statistical variance of the cluster density provided by the selected number of amplification cycles.

In some embodiments, a number of amplification cycles is selected for direct targeted sequencing by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; (g) sequencing the amplified surface-bound complements of the nucleic acid molecules to determine an average cluster density and an average cluster intensity after a predetermined number of sequencing cycles; (h) repeating steps (a)-(g) at a plurality of different numbers of amplification cycles; (i) selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected number of amplification cycles are within the predetermined cluster density range; and (j) selecting an the number of amplification cycles that provides the highest average cluster intensity from plurality of selected numbers of amplification cycles that provide an average cluster density that overlaps with a variance of the highest average cluster density or a cluster density variance that overlaps with the variance of the highest average cluster density. In some embodiments, the variance of the highest average cluster density is a predetermined percentage of the highest average cluster density. In some embodiments, the variance of the highest average cluster density is a predetermined statistical variance associated with the highest average cluster density. In some embodiments, the cluster density variance provided by the selected number of amplification cycles is a predetermined percentage of the average cluster density provided by the selected number of amplification cycles. In some embodiments, the cluster density variance provided by the selected number of amplification cycles is a predetermined statistical variance of the cluster density provided by the selected number of amplification cycles.

Selection of the number of amplification cycles can include repeating steps (a)-(g) at a plurality of amounts for one or more additional critical parameters (such as a plurality of amounts of the sequencing library or a plurality of numbers of amplification cycles), which can be selected sequentially or simultaneously.

In some embodiments, the plurality of different numbers of amplification cycles includes 2 or more different numbers of amplification cycles, 3 or more different numbers of amplification cycles, 5 or more different numbers of amplification cycles, 10 or more different numbers of amplification cycles, 25 or more different numbers of amplification cycles, or 50 or more different numbers of amplification cycles. In some embodiments, the different numbers of amplification cycles are within a predetermined range. In some embodiments, the different numbers of amplification cycles are evenly spaced or approximately evenly spaced within the range. In some embodiments, the different numbers of amplification cycles are unevenly spaced within the range.

In some embodiments, the number of amplification cycles is about 20 or more, about 25 or more, about 30 or more, about 35 or more, about 40 or more, about 45 or more, about 50 or more, about 60 or more, about 65 or more, about 70 or more, about 80 or more, or about 90 or more). In some embodiments, the number of amplification cycles is about 100 or less (such as about 90 or less, about 80 or less, about 70 or less, about 60 or less, about 50 or less, or about 40 or less). In some embodiments, the number of amplification cycles is any number of cycles, such as about 30, 31, 32, 33, 34, 35, 36, 37, 38, 39, 40, 41, 42, 43, 44, 45, 46, 47, 48, 49, or 50.

### Sequencing Metrics

The amounts of the critical parameters (e.g., the amount of the sequencing library, the amount of the capture probe library, or the number of amplification cycles) is selected based on one or more determined sequencing metrics (e.g., an average cluster density, a sequencing quality metric, or an average cluster intensity). Determination of the sequencing metrics is well known in the art.

### Sequencing Metrics—Cluster Density

The amounts of the critical parameters discussed herein are selected based on at least an average cluster density after a predetermined number of sequencing cycles. The capture probes in the capture probe library hybridize to surface-bound oligonucleotides. The surface-bound oligonucleotide is extended using the hybridized capture probe as a template to produce surface-bound capture probes. The surface-bound capture probe can then hybridize to nucleic acid molecules from the sequencing library, and the surface-bound capture probe can be extended using the nucleic acid molecules as a template to form surface-bound complements of the nucleic acid molecules. The surface-bound complements are amplified to form clusters, and the cluster density is related to at least the amount of the surface-bound complements that are successfully amplified (which, in turn, is related to the amount of capture probe library and the amount of sequencing library).

A target cluster density is often recommended by a sequencer manufacturer. However, due to the variables in direct targeted sequencing, it was previously found to be difficult to reach the target (or predetermined cluster density). Cluster density below the lower limit of the cluster density range occurs following the generation of too few clusters, or underclustering. Cluster density above the upper limit of the cluster density range occurs when clusters are too close together, and are overclustered. Cluster density below the upper limit of the predetermined cluster density range ensures diversity in the sequenced clusters while avoiding overclustering.

In some embodiments, the sequencing surface is divided into subsections, or “tiles.” An average cluster density from the cluster density of the tiles can be determined, as can a statistical variance (e.g., an interquartile range, a standard deviation, a dispersion, or any other similar statistical metric). If the sequencing surface is not divided into subsections, the “average cluster density” is considered the determined cluster density for the sequencing surface.

In some embodiments, the predetermined cluster density range is set within about 100 K/mm² to about 10,000 K/mm² (such as about 100 K/mm² to about 300 K/mm², about 300 K/mm² to about 700 K/mm², about 700 K/mm² to about 900 K/mm², about 900 K/mm² to about 1100 K/mm², about 1100 K/mm² to about 1300 K/mm², about 1300 K/mm² to about 1500 K/mm², about 1500 K/mm² to about 2000 K/mm², about 2000 K/mm² to about 3000 K/mm², about 3000 K/mm² to about 4000 K/mm², about 4000 K/mm² to about 5000 K/mm², about 5000 K/mm² to about 10,000 K/mm²). In some embodiments, the predetermined cluster density range is a range of any size from about 100 K/mm² to about 10,000 K/mm². In some embodiments, the predetermined cluster density range is a range of any size greater than about 100 K/mm² (such as about 300 K/mm² or more, about 500 K/mm² or more, about 1000 K/mm² or more, about 2000 K/mm² or more, about 5000 K/mm² or more). In some embodiments, the predetermined cluster density range is a range of any size of about 10,000 K/mm² or less (such as about 5000 K/mm² or less, about 2000 K/mm² or less, about 1000 K/mm² or less, about 500 K/mm² or less). In some embodiments, the predetermined cluster density range is a range of any size greater than about 10,000 K/mm².

In some embodiments, the highest average cluster density is about 100 K/mm² to about 10,000 K/mm² (such as about 100 K/mm² to about 300 K/mm², about 300 K/mm² to about 700 K/mm², about 700 K/mm² to about 900 K/mm², about 900 K/mm² to about 1100 K/mm², about 1100 K/mm² to about 1300 K/mm², about 1300 K/mm² to about 1500 K/mm², about 1500 K/mm² to about 2000 K/mm², about 2000 K/mm² to about 3000 K/mm², about 3000 K/mm² to about 4000 K/mm², about 4000 K/mm² to about 5000 K/mm², about 5000 K/mm² to about 10,000 K/mm²). In some embodiments, the highest average cluster density is greater than about 100 K/mm² (such as about 300 K/mm² or more, about 500 K/mm² or more, about 1000 K/mm² or more, about 2000 K/mm² or more, about 5000 K/mm² or more). In some embodiments, the highest average cluster density is less than about 10,000 K/mm² (such as about 5000 K/mm² or less, about 2000 K/mm² or less, about 1000 K/mm² or less, about 500 K/mm² or less). In some embodiments, the highest average cluster density is greater than about 10,000 K/mm².

In some embodiments, an amount of a critical parameter that provides the highest average cluster density, wherein the highest average cluster density is within a predetermined cluster density range, from among the plurality of amounts of the critical parameter is selected.

In some embodiments, an amount of the critical parameter or a plurality of amounts of the critical parameter is selected if the average cluster density provided by the amount or amounts of the critical parameter overlaps with a variance of the highest average cluster density, wherein the highest average cluster density and the average cluster density provided by the selected amount or amounts of the critical parameter are within a predetermined cluster density range. For example, the average cluster density provided by a plurality of amounts of the critical parameter can be determined, and the amount of the critical parameter that provides the highest average cluster density within the predetermined cluster density range is identified. A variance can be associated with the highest average cluster density. The variance can be, for example, a statistical variance, a predetermined percentage of the highest average cluster density, or above a predetermined percentile. The amount or amounts of the critical parameter that provides an average cluster density that overlaps (i.e., falls within) the variance associated with the highest average cluster density can be selected if the average cluster density for that amount or amounts is within the predetermined cluster density range.

In some embodiments, an amount of the critical parameter or a plurality of amounts of the critical parameter is selected if the amount or amounts provide a cluster density variance that overlaps with the variance associated with the highest average cluster density, wherein the highest average cluster density and the average cluster density provided by the selected amount of the critical parameter are within a predetermined cluster density range. For example, the average cluster density provided by a plurality of amounts of the critical parameter can be determined, and the amount of the critical parameter that provides the highest average cluster density within the predetermined cluster density range is identified. A variance can be associated with the highest average cluster density. The variance can be, for example, a statistical variance, a predetermined percentage of the highest average cluster density, or above a predetermined percentile. Similarly, the amount or amounts of the critical parameter can have a variance associate with the average cluster density for each amount, and the variance can be, for example, a statistical variance of the average cluster density for that amount or a predetermined percentage of the of the average cluster density for that amount. If the variance associated with an amount of the critical parameter overlaps with the variance associated with the highest average cluster density, then that amount can be selected, so long as he average cluster density provided by the selected amount or amounts of the critical parameter are within a predetermined cluster density range. The overlap need not be full overlap, but can be a partial overlap.

In some embodiments, the variance is a predetermined percentage less than the highest average cluster density, such as about 1% to about 100% (such as about 5%, about 10%, about 15%, about 20%, about 30%, about 40%, about 50%, about 60%, about 70%, about 80%, or about 90%). In some embodiments, the predetermined variance is any percentage, such as about 1%, about 2%, about 3%, about 4%, about 5%, about 6%, about 7%, about 8%, about 9%, about 10%, or more.

In some embodiments, the variance is a predetermined percentage less than the highest average cluster density, such as about 1% to about 100% (such as about 5%, about 10%, about 15%, about 20%, about 30%, about 40%, about 50%, about 60%, about 70%, about 80%, or about 90%). In some embodiments, the predetermined variance is any percentage, such as about 1%, about 2%, about 3%, about 4%, about 5%, about 6%, about 7%, about 8%, about 9%, about 10%, or more.

The cluster density is determined after a predetermined number of sequencing cycles. In some embodiments, the cluster density is determined after about 1 to about to about 100 sequencing cycles (such as about 1 to about 10, about 10 to about 20, about 20 to about 25, about 25 to about 30, about 30 to about 35, about 35 to about 40, about 40 to about 45, about 45 to about 50, about 50 to about 55, about 55 to about 60, about 60 to about 70, about 70 to about 80, about 80 to about 90, or about 90 to about 100 cycles). In some embodiments, the predetermined number of sequencing cycles is about 5 or higher (such as about 10 or higher, about 20 or higher, about 30 or higher, about 35 or higher, about 40 or higher, about 45 or higher, about 50 or higher, about 55 or higher, about 60 or higher, about 65 or higher, about 70 or higher, about 80 or higher, or about 90 or higher). In some embodiments, the predetermined number of sequencing cycles is about 100 or lower (such as about 90 or lower, about 80 or lower, about 70 or lower, about 60 or lower, about 50 or lower, about 40 or lower, about 30 or lower, about 20 or lower, or about 10 or lower). In some embodiments, the predetermined number of sequencing cycles is any number of cycles, such as about 20, 21, 22, 23, 24, 25, 26, 27, 28, 29, or 30 or more.

### Sequencing Metrics—Cluster Intensity

In some embodiments of the present invention, an average cluster intensity is measured after a predetermined number of sequencing cycles and can also be employed to select the amounts of one or more critical parameters. Next generation sequencers generally use an imager to capture cluster intensity after each sequencing cycle to determine an incorporation of a base (nucleotide) in each cluster. For example, each sequencing cycle can comprise incorporation of four fluorescently labeled nucleotides. Following laser excitation, an image is captured and the intensity is determined for each fluorescent label (or color) for each cluster.

In some embodiments, the intensity is calculated in the sequencing platform software (such as the SAV sequencing analysis viewer software). The cluster intensity can be, for example, a “corrected intensity” or a “called intensity.”

The cluster intensity is determined after a predetermined number of sequencing cycles. In some embodiments, the predetermined number of sequencing cycles is 1 to about 100 sequencing cycles (such as about 1 to about 10, about 10 to about 20, about 20 to about 25, about 25 to about 30, about 30 to about 35, about 35 to about 40, about 40 to about 45, about 45 to about 50, about 50 to about 55, about 55 to about 60, about 60 to about 70, about 70 to about 80, about 80 to about 90, or about 90 to about 100 cycles). In some embodiments, the predetermined number of sequencing cycles is about 5 or higher (such as about 10 or higher, about 20 or higher, about 30 or higher, about 35 or higher, about 40 or higher, about 45 or higher, about 50 or higher, about 55 or higher, about 60 or higher, about 65 or higher, about 70 or higher, about 80 or higher, or about 90 or higher). In some embodiments, the predetermined number of sequencing cycles is about 100 or lower (such as about 90 or lower, about 80 or lower, about 70 or lower, about 60 or lower, about 50 or lower, about 40 or lower, about 30 or lower, about 20 or lower, or about 10 or lower).

### Sequencing Metrics—Qualitative Sequencing Metric

Amounts of the critical parameters (e.g., amounts of the sequencing library, amounts of the capture probe library, and the number of amplification cycles) can also be based on an average qualitative sequencing metric. The qualitative sequencing metric is a value that quantifies sequencing quality. The qualitative sequencing metric can be, for example, a percent of clusters passing filter (often referred to as “% PF”) or a percent sequencing quality score (e.g., a “% Q10,” “% Q20,” or “% Q30”). In some embodiments, the sequencing quality metric is determined after a predetermined number of sequencing cycles, and the determined sequencing quality metric is used, in part, to select the amount of one or more critical parameters for direct targeted sequencing.

In some embodiments, the method comprises selecting the amount of the critical parameter that provides the highest average sequencing quality metric from a plurality of selected amounts of the critical parameter that provide an average cluster density that overlaps with a variance of the highest average cluster density or a cluster density variance that overlaps with the variance of the highest average cluster density, wherein he highest average cluster density and the average cluster densities provided by the plurality of selected amounts of the critical parameter are within a predetermined cluster density range.

In some embodiments, the method comprises selecting a plurality of amounts of the critical parameter that provide a sequencing quality metric above a predetermined threshold from the plurality of selected amounts of the critical parameter that provide an average cluster density that overlaps with a variance of the highest average cluster density or a cluster density variance that overlaps with the variance of the highest average cluster density, wherein he highest average cluster density and the average cluster densities provided by the plurality of selected amounts of the critical parameter are within a predetermined cluster density range. The predetermined threshold can be, for example, a predetermined percentage of the highest average sequencing quality metric below the highest average sequencing quality metric, a predetermined sequencing quality metric value, or a percentile. In some embodiments, the predetermined percentage of the highest average sequencing quality metric is about 1% to about 50% (such as about 1% to about 40%, about 5% to about 30%, about 10% to about 25% or about 25%). In some embodiments, the predetermined percentage is about 50% or less, about 40% or less, about 30% or less, about 25% or less, about 20% or less, about 15% or less, about 10% or less, or about 5% or less. In some embodiments, the percentile is about 50th percentile or higher, about 60th percentile or higher, about 70th percentile or higher, about 80th percentile or higher, about 85th percentile or higher, about 90th percentile or higher, or about 95th percentile or higher. The predetermined sequencing quality metric value depends on the specific sequencing quality metric used, as described herein.

In some embodiments, the method comprises selecting a plurality of amounts of the critical parameter that provide an average sequencing quality metric that overlaps with a variance of the highest average sequencing quality metric, or a sequencing quality metric variance that overlaps with the variance of the highest average sequencing quality metric, from the plurality of selected amounts of the sequencing library that provide an average cluster density that overlaps with a variance of the highest average cluster density or a cluster density variance that overlaps with the variance of the highest average cluster density. The average sequencing quality metric is the average based on one or more tiles of the sequencing surface. If the surface only includes a single tile, the average sequencing quality metric is the sequencing quality metric for that tile. From those amounts of the critical parameter that provide an average cluster density that of the critical parameter that provide an average cluster density that overlaps with a variance of the highest average cluster density or a cluster density variance that overlaps with the variance of the highest average cluster density, an average sequencing quality metric is determined. From the determined average sequencing quality metrics, the highest average sequencing quality metric can be determined, along with a variance associated with the highest average sequencing quality metric. In some embodiments, a variance of the sequencing quality metric is determined for the critical parameters for which an average an average sequencing quality metric is determined. In some embodiments, the variance is a statistical variance (e.g., a standard deviation, interquartile range, a statistical dispersion, or other statistical variance). The statistical variance can be determined, for example, based on the cluster density variation on the surface for the amount of the critical parameter. For example, some surfaces include a plurality of tiles, and a cluster density is determined for each tile. A statistical variance can be determined for the amount of the critical parameter that provided the highest average cluster density from the cluster density variance of the tiles. In some embodiments, the variance is percentage of (e.g., within 5% or less, within 10% or less, within 15% or less, or within 20% or less) the determined highest average cluster density. In some embodiments, the variance is a percentile (for example, 70th percentile or above, 80th percentile or above, or 90th percentile or above) for the average cluster densities in the pluralities of amounts of the critical parameters. In some embodiments, the selected plurality of amounts of the critical parameter provide an average sequencing quality metric that overlaps with the variance of the highest average sequencing quality metric (that is, the average sequencing quality metric provided by each of the selected amounts of the critical parameter are within the variance (e.g., statistical variance, percentage of, or percentile) of the highest average sequencing quality metric). In some embodiments, the selected plurality of amounts of the critical parameter have a variance (e.g., a statistical variance or a percentage of) associated with the determined average sequencing quality metric, and that variance overlaps with the variance associated with the highest average sequencing quality metric. The variances need not fully overlap as long as some portion of the variances overlap.

Methods for determining the sequencing quality score are known in the art (see for example, Illumina, Quality Scores for Next-Generation Sequencing, Technical Note: Informatics, Pub. No. 770-2021-058 (Apr. 23, 2014) available at www.illumina.com/content/dam/illumina-marketing/documents/products/technotes/technote Q-Scores.pdf). The sequencing quality score is determined using a Phred-like algorithm developed for assessing the quality of Sanger sequencing. A higher sequencing quality score indicates a smaller probability of error on a logarithmic scale. The percent sequencing quality score is the percentage of bases in a sequencing cycle that meet or surpass the sequencing quality score. For example, the sequencing quality score of 10 (Q10) indicates a probability of an incorrect base call of 1 in 10 (and an inferred base call accuracy of about 90%), and a % Q10 is the percentage of bases in the sequencing cycle that have an inferred base call accuracy of about 90% or greater. A quality score of 20 (Q20) indicates a probability of an incorrect base call of 1 in 100 (and an inferred base call accuracy of about 99%), and a % Q20 is the percentage of bases in the sequencing cycle that have an inferred base call accuracy of about 99% or greater. A quality score of 30 (Q30) indicates a probability of an incorrect base call of 1 in 1000 (and an inferred base call accuracy of about 99.9%), and a % Q30 is the percentage of bases in the sequencing cycle that have an inferred base call accuracy of about 99.9% or greater. The percent sequencing quality score is determined after a predetermined number of cycles using a predetermined sequencing quality score. In some embodiments, the sequencing quality metric is the percentage of bases with a sequencing quality score of about 10 to about 50 (i.e., Q10 to Q50) in a predetermined number of sequencing cycles (such as a sequencing quality score of about 10 or higher, about 15 or higher, about 20 or higher, about 25 or higher, about 30 or higher, about 35 or higher, about 40 or higher, about 45 or higher, or about 50).

In some embodiments, the sequencing quality metric is a percentage of clusters passing filter (% PF) after a predetermined number of cycles. Methods for determining a percentage of clusters passing filter is known in the art (see, for example, Illumina, Calculating Percent Passing Filter for Patterned and Nonpatterned Flow Cells, Technical Note: Informatics, Pub. No. 770-2014-043-B (2017), available at support.illumina.com/content/dam/illumine-marketing/documents/products/technotes/hiseq-x-percent-pf-technical-note-770-2014-043.pdf). In brief, the % PF is determined using a “chastity filter,” the ratio of the brightest base intensity divided by the sum of the first and second brightest base intensities. Clusters “pass filter” when no more than one base call has a chastity value of below a predetermined amount in a predetermined number of cycles. In some embodiments, the value for the chastity filter is set at between about 0.4 to about 1 (such as about 0.4 to about 0.5, about 0.5 to about 0.6, about 0.6 to about 0.7, about 0.7 to about 0.8, about 0.8 to about 0.9, or about 0.9 to about 1.0).

Other sequencing quality metrics are known in the art. For example, in some embodiments, the sequencing quality metric is a “% Perfect Reads,” defined as the percentage of reads that align perfectly, as determined by a spiked control sample. In some embodiments, the sequencing quality metric is the “Signal to Noise Ratio,” which is calculated as a mean called intensity divided by standard deviation of non-called intensities. In some embodiments, the sequencing quality metric is the “Full Width at Half Maximum” (FWHM), defined as the average full width of clusters at half maximum (in pixels). In some embodiments, the sequencing quality metric is the “% Base,” the percentage of clusters for which the selected base has been called. In some embodiments, the sequencing quality metric is the “Error Rate,” as determined by a spiked PhiX or other control sample. In some embodiments, the sequencing quality metric is the “% Aligned,” the percent of read aligning to PhiX or another control. In some embodiments, the sequencing quality metric is the “% Phasing” or “% Prephasing,” the percentage of molecules in a cluster for which sequencing falls behind (phasing) or jumps ahead (prephasing) of the current cycle within a read. In some embodiments, the sequencing quality metric is another sequencing quality metric. In some embodiments, the sequencing quality metric is the “Density Passing Filter,” the density of clusters passing filter (in thousands per mm²) after a predetermined number of cycles. In some embodiments, the sequencing quality metric is the “Density Passing Filter,” for each tile after a predetermined number of cycles.

One or more average sequencing quality metrics are determined after a predetermined number of sequencing cycles. In some embodiments, the average sequencing quality metric is determined after about 1 to about to about 100 sequencing cycles (such as about 1 to about 10, about 10 to about 20, about 20 to about 25, about 25 to about 30, about 30 to about 35, about 35 to about 40, about 40 to about 45, about 45 to about 50, about 50 to about 55, about 55 to about 60, about 60 to about 70, about 70 to about 80, about 80 to about 90, or about 90 to about 100 cycles). In some embodiments, the predetermined number of sequencing cycles is about 5 or higher (such as about 10 or higher, about 20 or higher, about 30 or higher, about 35 or higher, about 40 or higher, about 45 or higher, about 50 or higher, about 55 or higher, about 60 or higher, about 65 or higher, about 70 or higher, about 80 or higher, or about 90 or higher). In some embodiments, the predetermined number of sequencing cycles is about 100 or lower (such as about 90 or lower, about 80 or lower, about 70 or lower, about 60 or lower, about 50 or lower, about 40 or lower, about 30 or lower, about 20 or lower, or about 10 or lower). In some embodiments, the predetermined number of sequencing cycles is any number of cycles, such as about 20, 21, 22, 23, 24, 25, 26, 27, 28, 29, or 30 sequencing cycles.

### Direct Targeted Sequencing

The methods described herein are useful for selecting amounts of one or more critical parameters for direct targeted sequencing. The methods include enriching a sequencing library and sequencing the enriched sequencing library using a plurality of amounts of one or more critical parameters. The sequencing quality metrics can be determined from data collected during sequencing the enriched sequencing library. The sequencing library is enriched using capture probes. Capture probes from a capture probe library are designed to include sequence at one end that is complementary to the sequence of a surface-bound oligonucleotide, and a second sequence that comprises a portion of the region of interest.

The sequencing library is enriched and sequenced by (a) hybridizing capture probes in a capture probe library to surface-bound oligonucleotides, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a number of amplification cycles; and (g) sequencing the amplified surface-bound complements of the nucleic acid molecules.

FIG. 2 illustrates a flowchart for enriching and sequencing a sequencing library by direct targeted sequencing. At step 202, capture probes from a capture probe library are hybridized to surface-bound oligonucleotides. At step 204, following hybridization, the surface-bound oligonucleotides are extended, using the hybridized capture probe as a template, to produce surface-bound capture probes. At step 206, the capture probes are removed. At step 208, nucleic acid molecules from the sequencing library are hybridized to the surface-bound capture probes. At step 210, the surface-bound capture probes are extended using the nucleic acid molecules as a template, thereby producing surface-bound complements of the hybridized nucleic acid molecules. At step 212, the surface-bound complements of the nucleic acid molecules formed in step 210 are amplified by bridge amplification for a number of amplification cycles. At step 214, the amplified surface-bound complements of nucleic acid molecules are sequenced. Because the surface-bound complements are amplified to form copies of the surface-bound complements as well as complements of the surface-bound complements, it is understood that reference to sequencing amplified surface-bound complements can include sequencing the copies of the surface-bound complements and the copies of complements of the surface-bound complements.

Exemplary methods of direct targeted sequencing are described in U.S. Pat. No. 9,309,556, entitled “Direct Capture, Amplification and Sequencing of Target DNA using Immobilized Primers,” which is hereby incorporated by reference in its entirety. Additional exemplary methods of direct targeted sequencing are described in U.S. Pat. No. 9,092,401, entitled “System and Method for Detecting Genetic Variation”; Myllykangas et al. “Efficient targeted resequencing of human germline and cancer genomes by oligonucleotide-selective sequencing.” Nat Biotechnol. 29(11):1024-7 (2011); and Hopmans et al., “A programmable method for massively parallel targeted sequencing.” Nucleic Acids Res. 42(10):e88 (2014).

In direct targeted sequencing, one population of surface-bound oligonucleotides is derivatized following hybridization to capture probes from a capture probe library and extension to produce surface-bound capture probes. However, some amount of this population of surface-bound oligonucleotide necessarily remains unconverted to surface-bound capture probe in order to enable bridge amplification. If too many surface-bound capture probes are generated, there will be efficient target capture, but inefficient bridge amplification. If too few capture probes are generated, there will be inefficient capture of target sequences, but efficient amplification. Therefore, the ratio of sequencing library to capture probe library to surface-bound oligonucleotides is important for efficient direct targeted sequencing.

Direct targeted sequencing integrates target capture and sequencing on the same surface. A variety of solid support surface materials are known in the art, and non-limiting examples are described in U.S. Pat. No. 9,092,401. In some embodiments, the surface is a channel of a flow cell. In some embodiments, the surface is a sequencing flow cell. In some embodiments, the surface comprises a material that is reactive, such that under specified conditions, a molecule (such as an oligonucleotide or a nucleic acid molecule) can be attached directly to the surface. In some embodiments, the surface can be derivatized with proteins (such as enzymes, peptides) or with oligonucleotides by covalent or non-covalent bonding through one or more attachment sites, thereby immobilizing the protein or nucleic acid to the solid-support, or generating a “surface-bound” protein or nucleic acid. The term “surface-bound,” as used herein, refers to a nucleotide sequence that is immobilized to the surface. Immobilization can be accomplished through direct bonding of the nucleic acid to the solid support. Immobilization can also be accomplished through extension of immobilized nucleic acids using a hybridized template nucleic acid.

In some embodiments, the surface is subdivided into portions, or “lanes,” and in some embodiments, these portions are further subdivided into portions, or “tiles.” In some embodiments, sequencing occurs on a flow cell with multiple lanes (for example, 8 lanes). In some embodiments, each lane is subdivided into some number of tiles (e.g., 120 for GAIIx, 48 for HiSeq). In some embodiments, each lane has multiple samples, each with a unique nucleotide barcode sequence. In some embodiments, the cluster density, cluster intensity or other sequencing quality metric are determined relative to a portion of the surface. In some embodiments, the cluster density, cluster intensity or other sequencing quality metric are determined relative to a portion of the flow cell or other sequencing surface. In some embodiments, the portion of the surface is a “tile,” or subdivided region of the surface or imaging region. In some embodiments, the cluster density is the number of clusters (in thousands) per square millimeter of surface per tile. In some embodiments, the cluster intensity is the intensity per tile. In some embodiments, the value of another sequencing quality metric (such as % Q30 or % PF) is the value of the sequencing quality metric per tile.

Methods for enriching sequencing libraries using capture probes are generally known in the art, and can include hybrid capture methods (e.g., using biotinylated capture probes), PCR amplification using capture probes as PCR primers, and direct targeted sequencing. Capture probes comprise sequences that are complementary to a target nucleic acid sequence (e.g. a sequence comprising a portion of a “region of interest” or complementary to a “region of interest”) and can hybridize to a target nucleic acid sequence by the formation of hydrogen bonds between the complementary bases.

In direct targeted sequencing, capture probes from a capture probe library are hybridized to surface-bound oligonucleotides on a surface. The capture probes comprise a first end comprising a sequence that hybridizes to the surface-bound oligonucleotides and a second end comprising a portion of a region of interest. Following hybridization, the surface-bound capture probes are used as a template to extend the surface-bound oligonucleotides. The extension of surface-bound oligonucleotides produces surface-bound capture probes. These surface-bound capture probes comprise a sequence that is complementary to the sequence of the capture probe library, and is also complementary to the sequence of a portion of the region of interest, such that it can hybridize to the region of interest. The capture probes are then removed from surface-bound capture probes (e.g. by denaturation), resulting in surface-bound capture probes capable of hybridizing to a region of interest within a sequencing library.

Once the capture probes hybridize to the surface-bound oligonucleotides, the surface-bound oligonucleotides are extended using the capture probe as a template. This produces surface-bound capture probes comprising a sequence complementary to the portion of the region of interest, which can hybridize to nucleic acid molecules in the sequencing library that include the portion of the region of interest (or at least a sufficient amount of that portion to allow hybridization to the surface-bound capture probe). The sequence that hybridizes to the surface-bound oligonucleotides is preferably constant across all capture probes in the capture probe library, whereas the second end of the capture probe (which comprises a portion of the region of interest) can vary to hybridize to different portions of the region of interest. The capture probe library can include one or more identical copies of any given capture probe.

In some embodiments, the portion of the region of interest included in the capture probe is about 10 to about 300 bases in length (such as about 10 bases to about 20 bases, 20 bases to about 60 bases in length, about 60 bases to about 100 bases in length, or about 100 bases to about 160 bases, about 160 bases to about 220 bases, or about 220 bases to about 300 bases in length). The number of capture probes in the capture probe library can depend on the size of the region of interest, as a larger region of interest generally requires a larger number of capture probes for adequate coverage. In some embodiments, the capture probe library comprises about 10 or more unique capture probes (such as about 50 or more, about 100 or more, about 250 or more, about 500 or more, about 1000 or more, about 2500 or more, about 5000 or more, about 10,000 or more, about 25,000 or more, about 50,000 or more, about 100,000 or more, or about 200,000 or more) unique capture probes.

To enrich for regions of interest from within the sequencing library, the surface-bound capture probes are contacted with nucleic acid molecules from a sequencing library that comprises the region of interest. Nucleic acid molecules that comprise a portion of the sequence of the region of interest hybridize to the surface-bound capture probes. The nucleic acid molecules that hybridize to the surface-bound capture probes can be isolated from the non-hybridized nucleic acids, thereby enriching nucleic acids from the sequencing library for sequencing. Using the hybridized nucleic acid molecules as a template, the surface-bound capture probes are extended to produce surface-bound complements of the hybridized nucleic acid molecules.

The sequencing library comprises a plurality of nucleic acid molecules. In some embodiments the sequencing library comprises cell-free DNA (such as fetal cell-free DNA, tumor cell-free DNA, genomic cell-free DNA), fragmented DNA derived from cells in a sample (such as genomic DNA or mitochondrial DNA, which can be extracted from cells by lysing the cells and isolating the DNA contained therein). In some embodiments, the sequencing library comprises DNA extracted and isolated from cells within patient samples (such as blood, saliva, tissue samples, etc.). In some embodiments, the sequencing library is an RNA sequencing library, which can be reverse transcribed either before or after enrichment.

The sequencing library comprises the region of interest. The nucleic acid molecules in the sequencing library include genomic fragments from the sample, and at least a portion of the nucleic acid molecules in the sequencing library include a portion of the region of interest. As the region of interest can be smaller than the full genome, it is understood that at least a portion of the nucleic acids in the sequencing library can include a sequence other than from within the region of interest. In some embodiments, the nucleic acid molecules in the sequencing library are ligated to sequencing adapters (at one or both ends), which optionally include molecular barcodes or sample index barcodes. Sequencing library preparation for some sequencing platforms requires the addition of specific adapter sequences to the nucleic acids, which can be included in the sequencing adapters.

In some embodiments, the region of interest comprises one or more chromosomes. In some embodiments, the region of interest comprises one more non-coding regions in the genome (such as 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more regions). In some embodiments, the region of interest comprises one or more genes (such as 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more genes). In some embodiments, the region of interest comprises the exons of one or more genes (such as the exons from 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more genes). In some embodiments, the region of interest comprises one or more exons (such as 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more, 500 or more, 1000 or more, or 2000 or more exons). In some embodiments, the region of interest is contiguous.

In some embodiments, the region of interest in the sequencing library is about 10 to about 300 bases in length (such as about 10 bases to about 20 bases, 20 bases to about 60 bases in length, about 60 bases to about 100 bases in length, or about 100 bases to about 160 bases, about 160 bases to about 220 bases, or about 220 bases to about 300 bases in length). In some embodiments, the region of interest in the sequencing library comprises about 10 or more unique regions of interest (such as about 50 or more, about 100 or more, about 250 or more, about 500 or more, about 1000 or more, about 2500 or more, about 5000 or more, about 10,000 or more, about 25,000 or more, about 50,000 or more, about 100,000 or more, or about 200,000 or more) unique regions of interest.

In some embodiments, the region of interest is divided into one or more non-contiguous sub-regions. In some embodiments, the region of interest comprises a plurality of non-contiguous sub-regions of about 1 to about 1000 contiguous nucleotides (such as about 50 to about 100, about 100 to about 200, about 200 to about 300, about 400 to about 500, or about 500 to about 1000), at one or more positions within the sequencing library. In some embodiments, the plurality of non-contiguous sub-regions are of varying sizes within the range of about 1 to about 1000 nucleotides (such as varying sizes of about 50 to about 100, about 100 to about 200, about 200 to about 300, about 400 to about 500, and about 500 to about 1000). In some embodiments, the region of interest comprises one more non-contiguous sub-regions (such as 2 or more, 3 or more, 4 or more, 5 or more, 10 or more, 15 or more, 20 or more, 30 or more, 40 or more, 50 or more, 75 or more, 100 or more, 150 or more, 200 or more, or 250 or more regions).

The region of interest can be one or more bases, which need not be contiguous, at one or more positions within the genome. For example, in some embodiments, the region of interest comprises 1 or more non-contiguous positions, 2 or more non-contiguous positions, 3 or more non-contiguous positions, 4 or more non-contiguous positions, 5 or more non-contiguous positions, 10 or more non-contiguous positions, 25 or more non-contiguous positions, 50 or more non-contiguous positions, 100 or more non-contiguous positions, 150 or more non-contiguous positions, 200 or more non-contiguous positions, or 250 more non-contiguous positions. In some embodiments, each of the non-contiguous positions comprises 1 or more contiguous bases, 2 or more contiguous bases, 3 or more contiguous bases, 4 or more contiguous bases, or 5 or more contiguous bases. For example, in some embodiments each of the non-contiguous positions comprises 1 to about 20 contiguous bases (such as 1 to about 10 contiguous bases, or about 1 to about 5 contiguous bases).

In some embodiments, the sequencing library is fragmented to produce nucleic acid fragments. In some embodiments, the sequencing library is fragmented to produce nucleic acid fragments of between about 100 base pairs (bp) and about 2000 base pairs (such as about 100 bp to about 300 bp, about 300 to about 500 bp, about 500 to about 700 bp, about 700 to about 900 bp, about 900 to about 1100 bp, about 1100 bp to about 1300 bp, about 1300 bp to about 1500 bp, about 1500 bp to about 2000 bp). In some embodiments, the sequencing library is fragmented to produce nucleic acid fragments of more than about 100 base pairs (such as more than about 250 bp, more than about 500 bp, more than about 750 bp, more than about 1000 bp, or more than about 1500 bp). In some embodiments, the sequencing library is fragmented to produce nucleic acid fragments of less than about 2000 bp (such as less than about 1500 bp, less than about 1000 bp, less than about 750 bp, less than about 500 bp, or less than about 250 bp). In some embodiments, the sequencing library is end-repaired following fragmentation.

The surface can include a first population of surface-bound oligonucleotides and a second population of surface-bound oligonucleotides. The capture probe includes a first end comprising a sequence that hybridizes to the first population of surface-bound oligonucleotides, and the surface-bound capture probes are produced from the first population of surface-bound oligonucleotides. Since the surface-bound capture probes are extended using the hybridized nucleic acid molecules from the sequencing library to form the surface-bound complements of the nucleic acid molecules, the surface-bound complements of the nucleic acid molecules are also produced from the first population of surface-bound oligonucleotides. The surface-bound complements are amplified by bridge amplification, which relies on the surface-bound complements to hybridize to the second population of the surface-bound oligonucleotides at the unbound end of the surface-bound complements. To incorporate a sequence that hybridizes to the second population of surface-bound oligonucleotides, the nucleic acid molecules in the sequencing library can include a sequencing adapter, which includes a sequence of at least a portion of the second population of surface-bound oligonucleotides.

The surface-bound complements of the nucleic acid molecules are amplified by bridge amplification for a number of amplification cycles to form clusters. The production of clusters is dependent on several factors, including the number of amplification cycles. The term “bridge amplification” refers to a solid-phase polymerase chain reaction (PCR), in which the oligonucleotides (i.e., the surface-bound complements of the nucleic acid molecules) are bound to the surface by their 5′ ends. During amplification, the oligonucleotides form a “bridge” to other surface-bound oligonucleotides as they are extended. “Bridge amplification is known in the art, and further details are described in U.S. Pat. Nos. 9,092,401; 9,309,556; 7,115,400; 6,300,070; U.S. Patent Pub. No. 2014/0162278; U.S. Patent Pub. No. 2008/0286795; U.S. Patent Pub. No. 2008/0160580; Gudmundsson et al., Genome-wide association and replication studies identify four variants associated with prostate cancer susceptibility, Nat. Genet. vol. 41, pp. 1122-1126 (2009); and Turner et al., Massively parallel exon capture and library-free resequencing across 16 genomes, Nat. Methods, vol. 6, pp. 315-316 (2009).

Following bridge amplification, sequencing data is collected from the amplified surface-bound complements of the nucleic acid molecules to determine a cluster density and/or other sequencing metrics after a predetermined number of sequencing cycles. The amplification of complements of the nucleic acids comprising sequences that include a portion of the region of interest allows for the generation of sequencing data that is enriched for regions of interest, such as target genomic sequences, relative to non-target polynucleotides. Bridge amplification generates “clusters” of up to several thousand clonal copies of the surface-bound complements in close proximity on the surface. The cluster density is defined as the number of distinct clonal nucleic acid clusters (in the thousands, or “K”) present on the surface per millimeter squared (“mm²”). The cluster density has an impact on sequencing performance in terms of data quality and total data output quantity.

The amplified surface-bound complements of the nucleic acids can be sequenced using a high-throughput sequencer, such as an Illumina HiSeq2500. Other methods of sequencing are known in the art. The predetermined cluster density range depends on the sequencing instrument, sequencing mode the sequencing reagents used, and other factors. Guidelines for optimal cluster density ranges are often provided by the manufacturer of the sequencing instrument.

The highest intensity base incorporated into a cluster is recorded and its intensity is compared to the next highest fluorescent base recorded for the cluster. This information is used to calculate the chastity filter ratio, a quality control measure utilized to determine acceptance or rejection of individual clusters. The chastity filter ratio is derived by dividing the fluorescence of the highest fluorescent intensity base by the sum of the fluorescence of the highest fluorescent intensity base and the fluorescence of the next highest fluorescence intensity base. In some embodiments, a ratio of 0.6 or greater is considered a “passing” ratio. The chastity filter can remove clusters of low uniformity. The sequencing quality score, (Q score) is Q=−10 log₁₀(e). The Q score is logarithmically related to error probability (e) and is conceptually analogous to the Phred quality score used in Sanger sequencing. For example, bases with Q20 and Q30 scores have a 1:100 and 1:1000 probability of being called incorrectly. The chastity filter is a quality control measure utilized by Illumina to determine acceptance or rejection of individual clusters. This filter is typically applied after the first 25 sequencing cycles. For example, in Illumina Sequencing Analysis Viewer Software, the P90 A, C, G, and T metrics in the Imagine Tab Metrics Table can be used to show the intensity values extracted from each cluster during sequencing-by-synthesis. In this example, following each sequencing cycle, imagers capture intensity values at cluster locations in tiles, wherein each tile has a reference location on the flow cell. For example, in four-channel sequencing-by-synthesis, following each base addition (sequencing cycle) four images are collected from each tile (one for each of the four base dyes for nucleotides A, T, G, and C). The tile images constitute the raw data from which sequence data is derived.

### Methods for Direct Targeted Sequencing of a Test Sequencing Library

The selected amount of one or more critical parameters can be used to enrich and sequence a test sequencing library by direct targeted sequencing using the selected amount of the one or more critical parameters. For example, in some embodiments, there is provided a method of sequencing a test sequencing library, comprising (a) hybridizing capture probes in a capture probe library to surface-bound oligonucleotides using a selected amount of the capture probe library, the capture probes comprising a first end comprising a sequence that hybridizes to surface-bound oligonucleotides and a second end comprising a portion of a region of interest; (b) extending the surface-bound oligonucleotides using the hybridized capture probes as a template to produce surface-bound capture probes comprising a sequence that hybridizes to a portion of a region of interest; (c) removing the capture probes; (d) hybridizing nucleic acid molecules from a sequencing library comprising the region of interest to the surface-bound capture probes using a selected amount of the sequencing library; (e) extending the surface-bound capture probes using the hybridized nucleic acid molecules as a template to produce surface-bound complements of the nucleic acid molecules; (f) amplifying the surface-bound complements of the nucleic acid molecules by bridge amplification for a selected number of amplification cycles; (g) sequencing the amplified surface-bound complements of the nucleic acid molecules.

In some embodiments the test sequencing library comprises cell-free DNA (such as fetal cell-free DNA, tumor cell-free DNA, genomic cell-free DNA), fragmented DNA derived from cells in a sample (such as genomic DNA or mitochondrial DNA, which can be extracted from cells by lysing the cells and isolating the DNA contained therein). In some embodiments, the test sequencing library comprises DNA extracted and isolated from cells within patient samples (such as blood, saliva, tissue samples, etc.). In some embodiments, the test sequencing library is an RNA sequencing library, which can be reverse transcribed either before or after enrichment. In some embodiments the test sequencing library is enriched for target regions within the test sequencing library. In some embodiments the enriched test sequencing library is sequenced. In one aspect, the test sequencing library is enriched for target regions such that sequencing of the test sequencing library can be used for targeted genotyping, including targeting SNPs and indel variants. For example, test sequencing libraries derived from patient samples may sequenced to obtain information relating to a target region corresponding to a small portion of the genome, such as 100 to 200 genes that are related to more common genetic diseases.

In one aspect, the methods of the invention can be used to identify causal genetic variants within a test sequencing library. In general, causal genetic variants are genetic variants for which there is statistical, biological, and/or functional evidence of association with a disease or trait. A single causal genetic variant can be associated with more than one disease or trait. Non-limiting examples of types of causal genetic variants include single nucleotide polymorphisms (SNP), deletion/insertion polymorphisms (DIP), copy number variants (CNV), short tandem repeats (STR), restriction fragment length polymorphisms (RFLP), simple sequence repeats (SSR), variable number of tandem repeats (VNTR), randomly amplified polymorphic DNA (RAPD), amplified fragment length polymorphisms (AFLP), inter-retrotransposon amplified polymorphisms (IRAP), long and short interspersed elements (LINE/SINE), long tandem repeats (LTR), mobile elements, retrotransposon microsatellite amplified polymorphisms, retrotransposon-based insertion polymorphisms, sequence specific amplified polymorphism, and heritable epigenetic modification (for example, DNA methylation). A number of causal genetic variants are known in the art. Non-limiting examples of causal genetic variants are also described in US20100022406, “System and methods for detecting genetic variation,” which is hereby incorporated by reference in its entirety.

In some embodiments, the amount of the sequencing library is about 50 μg to about 500 μg (for example, about 75 μg to about 350 μg, about 100 μg to about 250 μg, about 125 μg to about 175 μg, or about 100 μg). In some embodiments, the amount of sequencing library is about 50 μg or more (such as about 75 μg or more, about 100 μg or more, about 125 μg or more, about 150 μg or more, or about 200 μg or more). In some embodiments, the amount of the sequencing library is about 500 μg or less (such as about 400 μg or less, about 350 μg or less, about 300 μg or less, about 250 μg or less, about 200 μg or less, or about 175 μg or less). In some embodiments, the amount of the sequencing library is about 1 μM to about 50 μM (for example, about 1 μM to about 5 μM, about 5 μM to about 10 μM, about 10 μM to about 20 μM, or about 20 μM to about 50 μM). In some embodiments, the amount of sequencing library is about 1 μM or more (such as about 2 μM or more, about 2 μM or more, about 3 μM or more, about 5 μM or more, about 7 μM or more, or about 10 μM or more). In some embodiments, the amount of the sequencing library is about 50 μM or less (such as about 40 μM or less, about 20 μM or less, or about 10 μM or less), In some embodiments, the number of amplification cycles is about 20 or more, about 25 or more, about 30 or more, about 35 or more, about 40 or more, about 45 or more, about 50 or more, about 60 or more, about 65 or more, about 70 or more, about 80 or more, or about 90 or more). In some embodiments, the number of amplification cycles is about 100 or less (such as about 90 or less, about 80 or less, about 70 or less, about 60 or less, about 50 or less, or about 40 or less). In some embodiments, the number of amplification cycles is any number of cycles, such as about 30, 31, 32, 33, 34, 35, 36, 37, 38, 39, 40, 41, 42, 43, 44, 45, 46, 47, 48, 49, or 50.

In some embodiments, the amount of the capture probe library is about 10 nM to about 250 nM (such as about 20 nM to about 200 nM, about 30 nM to about 150 nM, about 40 nM to about 100 nM, or about 50 nM to about 65 nM). In some embodiments, the amount of the capture probe library is about 10 nM or more (such as about 20 nM or more, about 30 nM or more, about 40 nM or more, or about 50 nM or more). In some embodiments, the amount of the capture probe library is about 250 nM or less (such as about 200 nM or less, about 150 nM or less, about 100 nM or less, about 75 nM or less, or about 65 nM or less). In some embodiments, the amount of the capture probe library is about 100 nanograms (ng) to about 1000 ng, about 150 ng to about 900 ng, about 250 ng to about 800 ng, about 300 ng to about 700 ng, about 400 ng to about 600 ng, or about 425 ng to about 550 ng). In some embodiments, the amount of the capture probe library is about 100 ng or more (such as about 150 ng or more, about 250 ng or more, about 300 ng or more, about 400 ng or more, or about 425 ng or more. In some embodiments, the amount of the capture probe library is about 1000 ng or less (such as about 900 ng or less, about 800 ng or less, about 700 ng or less, about 600 ng or less, about 550 ng or less, or about 500 ng or less).

### EXEMPLARY EMBODIMENTS

### Embodiment 1

A method for selecting an amount of a sequencing library for direct targeted sequencing, comprising:

(a) hybridizing capture probes in a capture probe library to surface-bound oligonucleotides, the capture probes comprising a first end comprising a sequence that hybridizes to surface-bound oligonucleotides and a second end comprising a portion of a region of interest;

(b) extending the surface-bound oligonucleotides using the hybridized capture probes as a template to produce surface-bound capture probes comprising a sequence that hybridizes to a portion of a region of interest;

(c) removing the capture probes;

(d) hybridizing nucleic acid molecules from a sequencing library comprising the region of interest to the surface-bound capture probes;

(e) extending the surface-bound capture probes using the hybridized nucleic acid molecules as a template to produce surface-bound complements of the nucleic acid molecules;

(f) amplifying the surface-bound complements of the nucleic acid molecules by bridge amplification for a number of amplification cycles;

(g) sequencing the amplified surface-bound complements of the nucleic acid molecules to determine an average cluster density after a predetermined number of sequencing cycles;

(h) repeating steps (a)-(g) at a plurality of different amounts of the sequencing library; and

(i) selecting an amount of the sequencing library that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected amount of the sequencing library are within a predetermined cluster density range; or
(3) a cluster density variance that overlaps with the variance of the highest average cluster density, wherein the highest average cluster density and the average cluster density provided by the selected amount of the sequencing library are within a predetermined cluster density range.

### Embodiment 2

The method of embodiment 1, wherein the variance of the highest average cluster density is a predetermined percentage of the highest average cluster density.

### Embodiment 3

The method of embodiment 1, wherein the variance of the highest average cluster density is a predetermined statistical variance associated with the highest average cluster density.

### Embodiment 4

The method of any one of embodiments 1-3, wherein the cluster density variance provided by the selected amount of the sequencing library is a predetermined percentage of the average cluster density provided by the selected amount of the sequencing library.

### Embodiment 5

The method of any one of embodiments 1-3, wherein the cluster density variance provided by the selected amount of the sequencing library is a predetermined statistical variance of the cluster density provided by the selected amount of the sequencing library.

### Embodiment 6

The method of any one of embodiments 1-5, comprising:

determining an average sequencing quality metric after the predetermined number of sequencing cycles;

selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within the predetermined cluster density range; and selecting the amount of the sequencing library that provides the highest average sequencing quality metric from the plurality of selected amounts of the sequencing library that provide an average cluster density that overlaps with a variance of the highest average cluster density or a cluster density variance that overlaps with the variance of the highest average cluster density.

### Embodiment 7

The method of any one of embodiments 1-5, further comprising:

determining an average cluster intensity and an average sequencing quality metric after the predetermined number of sequencing cycles;

selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range;

selecting a plurality of amounts of the sequencing library that provide an average sequencing quality metric that overlaps with a variance of the highest average sequencing quality metric, or a sequencing quality metric variance that overlaps with the variance of the highest average sequencing quality metric, from the plurality of selected amounts of the sequencing library that provide an average cluster density that overlaps with a variance of the highest average cluster density or a cluster density variance that overlaps with the variance of the highest average cluster density; and

selecting the amount of the sequencing library that provides the highest average cluster intensity from the plurality of selected amounts of the sequencing library that provide an average sequencing quality metric that overlaps with a variance of the highest average sequencing quality metric, or a sequencing quality metric variance that overlaps with the variance of the highest average sequencing quality metric.

### Embodiment 8

The method of embodiment 7, wherein the variance of the highest average sequencing quality metric is a predetermined percentage of the highest average sequencing quality metric.

### Embodiment 9

The method of embodiment 7, wherein the variance of the highest average sequencing quality metric is a predetermined statistical variance associated with the highest average sequencing quality metric.

### Embodiment 10

The method of any one of embodiments 7-9, wherein the sequencing quality metric variance provided by the selected amount of the sequencing library is a predetermined percentage of the average sequencing quality metric provided by the selected amount of the sequencing library.

### Embodiment 11

The method of any one of embodiments 7-9, wherein the sequencing quality metric variance provided by the selected amount of the sequencing library is a predetermined statistical variance of the sequencing quality metric provided by the selected amount of the sequencing library.

### Embodiment 12

The method of any one of embodiments 6-11, wherein the sequencing quality metric is a percentage Q30 quality score or a percentage of clusters passing filter.

### Embodiment 13

The method of any one of embodiments 1-5, comprising:

determining an average cluster intensity after the predetermined number of sequencing cycles;

selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range; and

selecting an the amount of the sequencing library that provides the highest average cluster intensity from plurality of selected amounts of the sequencing library that provide an average cluster density that overlaps with a variance of the highest average cluster density or a cluster density variance that overlaps with the variance of the highest average cluster density.

### Embodiment 14

The method of any one of embodiments 1-13, further comprising repeating steps (a)-(g) at a plurality of amounts of the capture probe library; and selecting an amount of the capture probe library that provides:

(1) the highest average cluster density, wherein the highest average cluster density is within a predetermined cluster density range;

(2) an average cluster density that overlaps with a variance of the highest average cluster density, wherein the highest average cluster density and the average cluster density provided by the selected amount of the capture probe library are within a predetermined cluster density range; or

(3) a cluster density variance that overlaps with the variance of the highest average cluster density, wherein the highest average cluster density and the average cluster density provided by the selected amount of the capture probe library are within a predetermined cluster density range.

### Embodiment 15

The method of embodiment 14, wherein the amount of the sequencing library and the amount of the capture probe library are selected simultaneously.

### Embodiment 16

The method of embodiment 14, wherein the amount of the sequencing library and the amount of the capture probe library are selected sequentially.

### Embodiment 17

The method of any one of embodiments 14-16, comprising:

determining an average sequencing quality metric after the predetermined number of sequencing cycles;

selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and

selecting the amount of the capture probe library that provides the highest average sequencing quality metric from the plurality of selected amounts of the capture library that provide an average cluster density that overlaps with the variance of the highest average cluster density or a cluster density variance that overlaps with the variance of the highest average cluster density.

### Embodiment 18

The method of any one of embodiments 14-16, comprising:

determining an average sequencing quality metric and an average cluster intensity after the predetermined number of sequencing cycles;

selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;

selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture library that provide an average cluster density that overlaps with the variance of the highest average cluster density or a cluster density variance that overlaps with the variance of the highest average cluster density; and

selecting the amount of the capture probe library that provides the highest average cluster intensity from the plurality of amounts of the capture probe library that provide an average sequencing quality metric that overlaps with a variance of the highest average sequencing quality metric, or a sequencing quality metric variance that overlaps with the variance of the highest average sequencing quality metric.

### Embodiment 19

The method of any one of embodiments 14-16, comprising:

determining an average cluster intensity after the predetermined number of sequencing cycles;

selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and

selecting the amount of the capture probe library that provides the highest average cluster intensity from the plurality of selected amounts of the capture library that provide an average cluster density that overlaps with the variance of the highest average cluster density or a cluster density variance that overlaps with the variance of the highest average cluster density.

### Embodiment 20

The method of any one of embodiments 1-19, comprising repeating steps (a)-(g) at a plurality different numbers of amplification cycles; and selecting the number of amplification cycles that provides:

(1) the highest average cluster density, wherein the highest average cluster density is within a predetermined cluster density range;

(2) an average cluster density that overlaps with a variance of the highest average cluster density, wherein the highest average cluster density and the average cluster density provided by the selected number of amplification cycles are within a predetermined cluster density range; or

(3) a cluster density variance that overlaps with the variance of the highest average cluster density, wherein the highest average cluster density and the average cluster density provided by the selected number of amplification cycles are within a predetermined cluster density range.

### Embodiment 21

The method of embodiment 20, wherein the amount of the sequencing library and the number of amplification cycles are selected simultaneously.

### Embodiment 22

The method of embodiment 20, wherein the amount of the sequencing library and the number of amplification cycles are selected sequentially.

### Embodiment 23

The method of embodiment 20, wherein the amount of the sequencing library, amount of the capture probe library, and number of amplification cycles are selected simultaneously.

### Embodiment 24

The method of embodiment 20, wherein the amount of the sequencing library, the amount of the capture probe library, and the number of amplification cycles are selected sequentially.

### Embodiment 25

The method of any one of embodiments 20-24, comprising:

determining an average sequencing quality metric after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and

selecting the number of amplification cycles that provides the highest average sequencing quality metric from the plurality of selected amounts of the capture library that provide an average cluster density that overlaps with the variance of the highest average cluster density or a cluster density variance that overlaps with the variance of the highest average cluster density.

### Embodiment 26

The method of any one of embodiments 20-24, comprising:

determining an average cluster intensity after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;

selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

### Embodiment 27

The method of any one of embodiments 20-24, comprising:

determining an average cluster intensity and an average sequencing quality metric after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;

selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and

selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

### Embodiment 28

The method of any one of embodiments 1-28, comprising sequencing the sequencing library by direct targeted sequencing using the selected amount of the sequencing library, the selected amount of the capture probe library, or the selected number of amplification cycles.

### Embodiment 29

A method for selecting an amount of a capture probe library for direct targeted sequencing, comprising:

(a) hybridizing capture probes in a capture probe library to surface-bound oligonucleotides, the capture probes comprising a first end comprising a sequence that hybridizes to surface-bound oligonucleotides and a second end comprising a portion of a region of interest;

(b) extending the surface-bound oligonucleotides using the hybridized capture probes as a template to produce surface-bound capture probes comprising a sequence that hybridizes to a portion of a region of interest;

(c) removing the capture probes;

(d) hybridizing nucleic acid molecules from a sequencing library comprising the region of interest to the surface-bound capture probes;

(e) extending the surface-bound capture probes using the hybridized nucleic acid molecules as a template to produce surface-bound complements of the nucleic acid molecules;

(f) amplifying the surface-bound complements of the nucleic acid molecules by bridge amplification for a number of amplification cycles;

(g) sequencing the amplified surface-bound complements of the nucleic acid molecules to determine a cluster density after a predetermined number of sequencing cycles;

(h) repeating steps (a)-(g) at a plurality of different amounts of the capture probe library; and

(i) selecting an amount of the capture probe library that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected amount of the capture probe library are within a predetermined cluster density range; or
(3) a cluster density variance that overlaps with the variance of the highest average cluster density, wherein the highest average cluster density and the average cluster density provided by the selected amount of the capture probe library are within a predetermined cluster density range.

### Embodiment 30

The method of embodiment 29, wherein the variance of the highest average cluster density is a predetermined percentage of the highest average cluster density.

### Embodiment 31

The method of embodiment 29, wherein the variance of the highest average cluster density is a predetermined statistical variance associated with the highest average cluster density.

### Embodiment 32

The method of any one of embodiments 29-31, wherein the cluster density variance provided by the selected amount of the capture probe library is a predetermined percentage of the average cluster density provided by the selected amount of the capture probe library.

### Embodiment 33

The method of any one of embodiments 29-31, wherein the cluster density variance provided by the selected amount of the capture probe library is a predetermined statistical variance of the cluster density provided by the selected amount of the capture probe library.

### Embodiment 34

The method of any one of embodiments 29-33, comprising:

determining an average sequencing quality metric after the predetermined number of sequencing cycles;

selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and

selecting the amount of the capture probe library that provides the highest average sequencing quality metric from the plurality of selected amounts of the capture library that provide an average cluster density that overlaps with the variance of the highest average cluster density or a cluster density variance that overlaps with the variance of the highest average cluster density.

### Embodiment 35

The method of any one of embodiments 29-33, comprising:

determining an average sequencing quality metric and an average cluster intensity after the predetermined number of sequencing cycles;

selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;

selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture library that provide an average cluster density that overlaps with the variance of the highest average cluster density or a cluster density variance that overlaps with the variance of the highest average cluster density; and

selecting the amount of the capture probe library that provides the highest average cluster intensity from the plurality of amounts of the capture probe library that provide an average sequencing quality metric that overlaps with a variance of the highest average sequencing quality metric, or a sequencing quality metric variance that overlaps with the variance of the highest average sequencing quality metric.

### Embodiment 36

The method of any one of embodiments 29-33, comprising:

determining an average cluster intensity after the predetermined number of sequencing cycles;

selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and

selecting the amount of the capture probe library that provides the highest average cluster intensity from the plurality of selected amounts of the capture library that provide an average cluster density that overlaps with the variance of the highest average cluster density or a cluster density variance that overlaps with the variance of the highest average cluster density.

### Embodiment 37

The method of any one of embodiments 29-36, comprising repeating steps (a)-(g) at a plurality different numbers of amplification cycles; and selecting the number of amplification cycles that provides:

(1) the highest average cluster density, wherein the highest average cluster density is within a predetermined cluster density range;

(2) an average cluster density that overlaps with a variance of the highest average cluster density, wherein the highest average cluster density and the average cluster density provided by the selected number of amplification cycles are within a predetermined cluster density range; or

(3) a cluster density variance that overlaps with the variance of the highest average cluster density, wherein the highest average cluster density and the average cluster density provided by the selected number of amplification cycles are within a predetermined cluster density range.

### Embodiment 38

The method of embodiment 37, wherein the amounts of the capture probe library and the number of amplification cycles are selected simultaneously.

### Embodiment 39

The method of embodiment 37, wherein the amount of the capture probe library and the number of amplification cycles are selected sequentially.

### Embodiment 40

The method of any one of embodiments 37-39, comprising:

determining an average sequencing quality metric after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and

selecting the number of amplification cycles that provides the highest average sequencing quality metric from the plurality of selected numbers of amplification cycles that provide an average cluster density that overlaps with the variance of the highest average cluster density or a cluster density variance that overlaps with the variance of the highest average cluster density.

### Embodiment 41

The method of any one of embodiments 37-39, comprising:

determining an average cluster intensity after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;

selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

### Embodiment 42

The method of any one of embodiments 37-39, comprising:

determining an average cluster intensity and an average sequencing quality metric after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range;

selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and

selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

### Embodiment 43

The method of any one of embodiments 29-42, comprising sequencing the sequencing library by direct targeted sequencing using the selected amount of the capture probe library or the selected number of amplification cycles.

### Embodiment 44

A method for selecting a number of amplification cycles for direct targeted sequencing, comprising:

(a) hybridizing capture probes in a capture probe library to surface-bound oligonucleotides, the capture probes comprising a first end comprising a sequence that hybridizes to surface-bound oligonucleotides and a second end comprising a portion of a region of interest;

(b) extending the surface-bound oligonucleotides using the hybridized capture probes as a template to produce surface-bound capture probes comprising a sequence that hybridizes to a portion of a region of interest;

(c) removing the capture probes;

(d) hybridizing nucleic acid molecules from a sequencing library comprising the region of interest to the surface-bound capture probes;

(e) extending the surface-bound capture probes using the hybridized nucleic acid molecules as a template to produce surface-bound complements of the nucleic acid molecules;

(f) amplifying the surface-bound complements of the nucleic acid molecules by bridge amplification for a number of amplification cycles;

(g) sequencing the amplified surface-bound complements of the nucleic acid molecules to determine a cluster density after a predetermined number of sequencing cycles;

(h) repeating steps (a)-(g) at a plurality of different numbers of amplification cycles;

and

(i) selecting a number of amplification cycles that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected number of amplification cycles are within a predetermined cluster density range; or
(3) a cluster density variance that overlaps with the variance of the highest average cluster density, wherein the highest average cluster density and the average cluster density provided by the selected number of amplification cycles are within a predetermined cluster density range.

### Embodiment 45

The method of embodiment 44, wherein the variance of the highest average cluster density is a predetermined percentage of the highest average cluster density.

### Embodiment 46

The method of embodiment 44, wherein the variance of the highest average cluster density is a predetermined statistical variance associated with the highest average cluster density.

### Embodiment 47

The method of any one of embodiments 44-46, wherein the cluster density variance provided by the selected number of sequencing cycles is a predetermined percentage of the average cluster density provided by the selected number of sequencing cycles.

### Embodiment 48

The method of any one of embodiments 44-46, wherein the cluster density variance provided by the selected number of sequencing cycles is a predetermined statistical variance of the cluster density provided by the selected number of sequencing cycles.

### Embodiment 49

The method of any one of embodiments 44-48, comprising:

determining an average sequencing quality metric after the predetermined number of sequencing cycles; and

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and

selecting the number of amplification cycles that provides the highest average sequencing quality metric from the plurality of selected numbers of amplification cycles that provide an average cluster density that overlaps with the variance of the highest average cluster density or a cluster density variance that overlaps with the variance of the highest average cluster density.

### Embodiment 50

The method any one of embodiments 44-48, comprising:

determining an average cluster intensity after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;

selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

### Embodiment 51

The method any one of embodiments 44-48, comprising:

determining an average cluster intensity and an average sequencing quality metric after the predetermined number of sequencing cycles;

selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range;

selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and

selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

### Embodiment 52

The method of any one of embodiments 44-51, comprising sequencing the sequencing library by direct targeted sequencing using the selected number of amplification cycles.

### Embodiment 53

The method of any one of embodiments 34, 35, 40, 42, 48, and 50 wherein the sequencing quality metric is a percentage Q30 quality score or a percentage of clusters passing filter.

### Embodiment 54

A method of sequencing a test sequencing library, comprising:

(a) hybridizing capture probes to surface-bound oligonucleotides, the capture probes comprising a first end comprising a sequence that hybridizes to the first population of surface-bound oligonucleotides and a second end comprising a sequence that hybridizes to a portion of a region of interest, wherein the concentration of the capture probes is about 40 to about 70 nanomolar;

(b) extending the surface-bound oligonucleotides using the hybridized capture probes as a template to produce surface-bound capture probes;

(c) removing the capture probes;

(d) hybridizing nucleic acid molecules from about 1 μM to about 50 μM of the test sequencing library comprising the region of interest to the surface-bound capture probes, wherein the concentration of the nucleic acid molecules results in a cluster density of about 600 K/mm² to about 1500 K/mm²;

(e) extending the surface-bound capture probes using the hybridized nucleic acid molecules as a template to produce surface-bound complements of the nucleic acid molecules;

(f) amplifying the surface-bound complements of the nucleic acid molecules by bridge amplification for at least 30 amplification cycles;

(g) sequencing the amplified surface-bound complements of the nucleic acid molecules.

### EXAMPLES

### Example 1

12,808 different capture probes in a capture probe library were hybridized to a first lane and a second lane of a HiSeq Paired-End Flow Cell v2 (Illumina catalog no. 15053059) using the same concentration of capture probe library for each lane. Probes on the surface of the sequencing plate were extended using the capture probes as a template, and the capture probes were removed. These steps resulted in surface-bound capture probes fixed to the plate at the same density in each lane. A sequencing library was then hybridized to the surface-bound capture probes in the first lane and the second lane, although the concentration of the sequencing library hybridized to the surface-bound capture probes in the second lane was 1/5 the concentration of the sequencing library hybridized to the surface-bound capture probes in the first lane. The surface-bound capture probes were extended using the hybridized nucleic acid molecules from the sequencing library as a template, and nucleic acid molecules un-bound to the surface were washed away. The surface bound nucleic acid molecules were amplified by bridge amplification, and the amplicons were sequenced using an Illumina HiSeq 2500 sequencer. Determined cluster density, clusters passing filter (% PF), percentage phasing, percentage prephasing, the number of reads, the number of reads passing filter (PF), percentage of bases with a quality score of 30 or higher (% Q30), and total yield is shown in Table 1.

TABLE 1

| | Density | Cluster PF | Phasing | Prephasing | Reads | Reads | % | Yield |
|---|---|---|---|---|---|---|---|---|
| Lane | (K/mm²) | (%) | (%) | (%) | (M) | PF (M) | ≥Q30 | (G) |
| 1 | 866 ± 93 | 91.27 ± 2.69 | 0.469 | 0.104 | 159.63 | 145.36 | 93.0 | 7.1 |
| 2 | 259 ± 49 | 73.13 ± 42.56 | 0.484 | 0.106 | 47.78 | 35.25 | 97.4 | 1.7 |

### Example 2

12,808 different capture probes in a capture probe library were hybridized to a first lane and a second lane of a HiSeq Paired-End Flow Cell v2 (Illumina catalog no. 15053059). The concentration of capture probe library used in the second lane was 1/5 the concentration of the capture probe library used in the first lane. Probes on the surface of the sequencing plate were extended using the capture probes as a template, and the capture probes were removed. These steps resulted in surface-bound capture probes fixed to the plate at the same density in each lane. A sequencing library was then hybridized to the surface-bound capture probes in the first lane and the second lane at the same concentration. The surface-bound capture probes were extended using the hybridized nucleic acid molecules from the sequencing library as a template, and nucleic acid molecules un-bound to the surface were washed away. The surface bound nucleic acid molecules were amplified by bridge amplification, and the amplicons were sequenced using an Illumina HiSeq 2500 sequencer. Determined cluster density, clusters passing filter (% PF), percentage phasing, percentage prephasing, the number of reads, the number of reads passing filter (PF), percentage of bases with a quality score of 30 or higher (% Q30), and total yield is shown in Table 2.

TABLE 2

| | Density | Cluster PF | Phasing | Prephasing | Reads | Reads | % | Yield |
|---|---|---|---|---|---|---|---|---|
| Lane | (K/mm²) | (%) | (%) | (%) | (M) | PF (M) | ≥Q30 | (G) |
| 1 | 750 ± 96 | 93.82 ± 2.04 | 0.316 | 0.116 | 138.28 | 129.47 | 95.7 | 6.3 |
| 2 | 248 ± 91 | 97.86 ± 0.07 | 0.324 | 0.113 | 45.71 | 44.67 | 98.3 | 2.2 |

1.-53. (canceled)

54. A method of sequencing a test sequencing library, comprising:
(a) hybridizing capture probes in a capture probe library to surface-bound oligonucleotides, the capture probes comprising a first end comprising a sequence that hybridizes to surface-bound oligonucleotides and a second end comprising a portion of a region of interest, wherein the concentration of the capture probes is about 40 to about 70 nanomolar;
(b) extending the surface-bound oligonucleotides using the hybridized capture probes as a template to produce surface-bound capture probes comprising a sequence that hybridizes to a portion of a region of interest;
(c) removing the capture probes;
(d) hybridizing nucleic acid molecules from about 1 μM to about 50 μM of a test sequencing library comprising the region of interest to the surface-bound capture probes, wherein the concentration of the nucleic acid molecules results in a cluster density of about 600 K/mm² to about 1500 K/mm²;
(e) extending the surface-bound capture probes using the hybridized nucleic acid molecules as a template to produce surface-bound complements of the nucleic acid molecules;
(f) amplifying the surface-bound complements of the nucleic acid molecules by bridge amplification for at least 30 amplification cycles;
(g) sequencing the amplified surface-bound complements of the nucleic acid molecules.

55.-59. (canceled)

60. A method for selecting an amount of a sequencing library for direct targeted sequencing, comprising sequencing a test sequencing library according to claim 54, wherein step (g) comprises sequencing the amplified surface-bound complements of the nucleic acid molecules to determine an average cluster density after a predetermined number of sequencing cycles, and wherein the method further comprises:
(h) repeating steps (a)-(g) at a plurality of different amounts of the sequencing library; and
(i) selecting an amount of the sequencing library that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected amount of the sequencing library are within a predetermined cluster density range; or
(3) a cluster density variance that overlaps with the variance of the highest average cluster density, wherein the highest average cluster density and the average cluster density provided by the selected amount of the sequencing library are within a predetermined cluster density range.

61. The method of claim 60, comprising:
determining an average sequencing quality metric after the predetermined number of sequencing cycles;
selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within the predetermined cluster density range; and
selecting the amount of the sequencing library that provides the highest average sequencing quality metric from the plurality of selected amounts of the sequencing library that provide an average cluster density that overlaps with a variance of the highest average cluster density or a cluster density variance that overlaps with the variance of the highest average cluster density.

62. The method of claim 60, further comprising:
determining an average cluster intensity and an average sequencing quality metric after the predetermined number of sequencing cycles;
selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range;
selecting a plurality of amounts of the sequencing library that provide an average sequencing quality metric that overlaps with a variance of the highest average sequencing quality metric, or a sequencing quality metric variance that overlaps with the variance of the highest average sequencing quality metric, from the plurality of selected amounts of the sequencing library that provide an average cluster density that overlaps with a variance of the highest average cluster density or a cluster density variance that overlaps with the variance of the highest average cluster density; and
selecting the amount of the sequencing library that provides the highest average cluster intensity from the plurality of selected amounts of the sequencing library that provide an average sequencing quality metric that overlaps with a variance of the highest average sequencing quality metric, or a sequencing quality metric variance that overlaps with the variance of the highest average sequencing quality metric.

63. The method of claim 60, comprising:
determining an average cluster intensity after the predetermined number of sequencing cycles;
selecting a plurality of amounts of the sequencing library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the sequencing library are within a predetermined cluster density range; and
selecting an the amount of the sequencing library that provides the highest average cluster intensity from plurality of selected amounts of the sequencing library that provide an average cluster density that overlaps with a variance of the highest average cluster density or a cluster density variance that overlaps with the variance of the highest average cluster density.

64. The method of claim 60, further comprising repeating steps (a)-(g) at a plurality of amounts of the capture probe library; and selecting an amount of the capture probe library that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected amount of the capture probe library are within a predetermined cluster density range; or
(3) a cluster density variance that overlaps with the variance of the highest average cluster density, wherein the highest average cluster density and the average cluster density provided by the selected amount of the capture probe library are within a predetermined cluster density range.

65. The method of claim 64, comprising:
determining an average sequencing quality metric after the predetermined number of sequencing cycles;
selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and
selecting the amount of the capture probe library that provides the highest average sequencing quality metric from the plurality of selected amounts of the capture library that provide an average cluster density that overlaps with the variance of the highest average cluster density or a cluster density variance that overlaps with the variance of the highest average cluster density.

66. The method of claim 64, comprising:
determining an average sequencing quality metric and an average cluster intensity after the predetermined number of sequencing cycles;
selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;
selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture library that provide an average cluster density that overlaps with the variance of the highest average cluster density or a cluster density variance that overlaps with the variance of the highest average cluster density; and
selecting the amount of the capture probe library that provides the highest average cluster intensity from the plurality of amounts of the capture probe library that provide an average sequencing quality metric that overlaps with a variance of the highest average sequencing quality metric, or a sequencing quality metric variance that overlaps with the variance of the highest average sequencing quality metric.

67. The method of claim 64, comprising:
determining an average cluster intensity after the predetermined number of sequencing cycles;
selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and
selecting the amount of the capture probe library that provides the highest average cluster intensity from the plurality of selected amounts of the capture library that provide an average cluster density that overlaps with the variance of the highest average cluster density or a cluster density variance that overlaps with the variance of the highest average cluster density.

68. The method of claim 60, comprising repeating steps (a)-(g) at a plurality different numbers of amplification cycles; and selecting the number of amplification cycles that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected number of amplification cycles are within a predetermined cluster density range; or
(3) a cluster density variance that overlaps with the variance of the highest average cluster density, wherein the highest average cluster density and the average cluster density provided by the selected number of amplification cycles are within a predetermined cluster density range.

69. The method of claim 68, comprising:
determining an average sequencing quality metric after the predetermined number of sequencing cycles;
selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and
selecting the number of amplification cycles that provides the highest average sequencing quality metric from the plurality of selected amounts of the capture library that provide an average cluster density that overlaps with the variance of the highest average cluster density or a cluster density variance that overlaps with the variance of the highest average cluster density.

70. The method of claim 68, comprising:
determining an average cluster intensity after the predetermined number of sequencing cycles;
selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;
selecting the number of amplification cycles that provides the highest average cluster intensity from the plurality of selected numbers of amplification cycles that provide an average cluster density that overlaps with the variance of the highest average cluster density or a cluster density variance that overlaps with the variance of the highest average cluster density.

71. The method of claim 68, comprising:
determining an average cluster intensity and an average sequencing quality metric after the predetermined number of sequencing cycles;
selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;
selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and
selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.

72. The method of claim 60, comprising sequencing the sequencing library by direct targeted sequencing using the selected amount of the sequencing library, the selected amount of the capture probe library, or the selected number of amplification cycles.

73. A method for selecting an amount of a capture probe library for direct targeted sequencing, comprising sequencing a test sequencing library according to claim 54, wherein step (g) comprises sequencing the amplified surface-bound complements of the nucleic acid molecules to determine a cluster density after a predetermined number of sequencing cycles, and wherein the method further comprises:
(h) repeating steps (a)-(g) at a plurality of different amounts of the capture probe library; and
(i) selecting an amount of the sequencing library that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected amount of the capture probe library are within a predetermined cluster density range; or
(3) a cluster density variance that overlaps with the variance of the highest average cluster density, wherein the highest average cluster density and the average cluster density provided by the selected amount of the capture probe library are within a predetermined cluster density range.

74. The method of claim 73, comprising:
determining an average sequencing quality metric after the predetermined number of sequencing cycles;
selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range; and
selecting the amount of the capture probe library that provides the highest average sequencing quality metric from the plurality of selected amounts of the capture library that provide an average cluster density that overlaps with the variance of the highest average cluster density or a cluster density variance that overlaps with the variance of the highest average cluster density.

75. The method of claim 73, comprising:
determining an average sequencing quality metric and an average cluster intensity after the predetermined number of sequencing cycles;
selecting a plurality of amounts of the capture probe library that provide an average cluster density that overlaps with a variance of the highest average cluster density, or a cluster density variance that overlaps with the variance of the highest average cluster density, wherein the highest average cluster density and the average cluster densities provided by the plurality of selected amounts of the capture probe library are within the predetermined cluster density range;
selecting a plurality of amounts of the capture probe library that provide an average sequencing quality metric that overlaps with a variance of the highest average sequencing quality metric, or a sequencing quality metric variance that overlaps with the variance of the highest average sequencing quality metric, from the plurality of selected amounts of the capture library that provide an average cluster density that overlaps with the variance of the highest average cluster density or a cluster density variance that overlaps with the variance of the highest average cluster density; and
selecting the amount of the capture probe library that provides the highest average cluster intensity from the plurality of amounts of the capture probe library that provide an average sequencing quality metric that overlaps with a variance of the highest average sequencing quality metric, or a sequencing quality metric variance that overlaps with the variance of the highest average sequencing quality metric.

76. A method for selecting a number of amplification cycles for direct targeted sequencing, comprising sequencing a test sequencing library according to claim 54, wherein step (g) comprises sequencing the amplified surface-bound complements of the nucleic acid molecules to determine a cluster density after a predetermined number of sequencing cycles, and wherein the method further comprises:
(h) repeating steps (a)-(g) at a plurality of different numbers of amplification cycles; and
(i) selecting an amount of the sequencing library that provides:
(1) the highest average cluster density, wherein the highest average cluster density is within a predetermined cluster density range;
(2) an average cluster density that overlaps with a variance of the highest average cluster density, wherein the highest average cluster density and the average cluster density provided by the selected numbers of amplification cycles are within a predetermined cluster density range; or
(3) a cluster density variance that overlaps with the variance of the highest average cluster density, wherein the highest average cluster density and the average cluster density provided by the selected amount of the sequencing library are within a predetermined cluster density range.

77. The method of claim 76, comprising:
determining an average sequencing quality metric after the predetermined number of sequencing cycles; and
selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range; and
selecting the number of amplification cycles that provides the highest average sequencing quality metric from the plurality of selected numbers of amplification cycles that provide an average cluster density that overlaps with the variance of the highest average cluster density or a cluster density variance that overlaps with the variance of the highest average cluster density.

78. The method of claim 76, comprising:
determining an average cluster intensity and an average sequencing quality metric after the predetermined number of sequencing cycles;
selecting a plurality of numbers of amplification cycles that provide an average cluster density that overlaps with a variance of the highest average cluster density, or a cluster density variance that overlaps with the variance of the highest average cluster density, wherein the highest average cluster density and the average cluster densities provided by the plurality of selected numbers of amplification cycles are within the predetermined cluster density range;
selecting a plurality of numbers of amplification cycles that provide an average sequencing quality metric that overlaps with a variance of the highest average sequencing quality metric, or a sequencing quality metric variance that overlaps with the variance of the highest average sequencing quality metric, from the plurality of selected numbers of amplification cycles that provide an average cluster density that overlaps with the variance of the highest average cluster density or a cluster density variance that overlaps with the variance of the highest average cluster density; and
selecting the number of amplification cycles that provide the highest average cluster intensity from the plurality of numbers of amplification cycles that provide an average sequencing quality metric that overlaps with a variance of the highest average sequencing quality metric, or a sequencing quality metric variance that overlaps with the variance of the highest average sequencing quality metric.
